(19) United States
(12) Patent Application Publication
(10) Pub. No.: US 2024/0050527 A1
(43) Pub. Date: Feb. 15, 2024

(54) METHODS OF TREATING AGE-RELATED MACULAR DISEASES USING AIMP2-DX2 AND OPTIONALLY A TARGET SEQUENCE FOR miR-142 AND COMPOSITIONS THEREOF

(71) Applicant: Generoath Co., Ltd., Seoul (KR)

(72) Inventors: Jin Woo CHOI, Seoul (KR); Kyunghwa BAEK, Gangneung-si, Gangwon-do (KR)

(21) Appl. No.: 18/246,578

(22) PCT Filed: Sep. 30, 2021
(86) PCT No.: PCT/IB2021/059018

§ 371 Date: Mar. 24, 2023

Related U.S. Application Data
(60) Provisional application No. 63/085,922, filed on Sep. 30, 2020.

(51) Int. Cl. A61K 38/17; A61K 48/00; A61P 27/02

(52) U.S. Cl. CPC A61K 38/1761; A61K 48/0058; A61K 48/0075; A61P 27/02; A61K 48/0066; C12N 2750/14143

(57) ABSTRACT

Disclosed herein are methods of treating age-related macular diseases, comprising administering to a subject in need thereof a vector comprising AIMP2-DX2 and optionally a target sequence for miR-142.

### CROSS-REFERENCE TO RELATED APPLICATIONS

This application claims benefit of the filing date of U.S. application Ser. No. 63/085,922, filed Sep. 30, 2020, the disclosure of which is incorporated herein by reference in its entirety.

### REFERENCE TO SEQUENCE LISTING SUBMITTED ELECTRONICALLY

The content of the electronically submitted sequence listing in ASCII text file (Name: 2493-0005W001 Sequence Listing ST25.txt; Size: 28 KB; and Date of Creation: Sep. 30, 2021) filed with the application is incorporated herein by reference in its entirety.

### FIELD OF THE INVENTION

Disclosed herein are methods of treating age-related macular diseases, comprising administering to a subject in need thereof a vector comprising AIMP2-DX2 and optionally a target sequence for miR-142.

### BACKGROUND OF THE INVENTION

Age-related macular disease (AMD) is the leading cause of vision loss in Europe, the United States and Australia. Almost two-thirds of the population over 80 years of age will have signs of AMD resulting from the wet or exudative form, which is characterized by the presence of drusen and CNV (subfoveal choroidal neovascularization). AMD is a chronic progressive disease characterized by damage to the central retina zone. Changes in choriocapillaries, retinal pigment epithelium (RPE), and Bruch's membrane (typical for aging) underlie AMD pathogenesis; however, the mechanisms launching the transfer of typical age-related changes in the pathological process are unknown. The death of photoreceptors and irreversible loss of vision become the results of pathological changes in RPE and choroid. Recent studies demonstrated that not only apoptotic but also autophagic and necrotic signaling cascades are involved in the cellular death of retinal cells (Telegina 2017).

“Dry” and “wet” forms of the disease are categorized. Approximately 90% cases are of the “dry” atrophic form of AMD; today, there is no method of its treatment. In the “dry” form of AMD, drusen are diagnosed in the macular area, pigment redistribution occurs, defects of pigment epithelium and the choriocapillary layer appear, and death of photoreceptors occurs against a background of RPE cell atrophy. “Wet” (exudative) form develops in ˜10% of AMD patients and is characterized by ingrowing of newly generated vessels through Bruch's membrane defects under the retinal pigment epithelium or under neuroepithelium. The increased permeability of newly generated vessels results in retinal edema, exudation, and hemorrhage in the vitreous body and retina (which finally becomes the reason for vision loss).

Currently, the laser-induced Bruch's membrane photocoagulation model is the most widely accepted and most frequently used experimental mouse CNV model. The model described here consists of the laser impact rupturing of Bruch's membrane, which leads to the growth of new blood vessels from the choroid into the subretinal space, mimicking the main characteristics of the exudative form of human AMD and offering the opportunity to explore the molecular mechanism of CNV through the use of a large panel of transgenic mice. The model has proven to be suitable for testing the efficacy of new drugs through systemic or local (intraocular) administration and has shown predictive value for drug effects in patients with AMD, for example, with vascular endothelial growth factor receptor (VEGFR) trap or anecortave acetate (Telegina 2017).

There are three main drugs that provide indirect anti-angiogenesis by blocking vascular endothelial growth factor (VEGF) in the retina. Ranibizumab (Lucentis, Genentech Inc., South San Francisco CA; commercialized worldwide by Novartis) was approved by the Food and Drug Administration (FDA) in 2006 for the treatment of neovascular AMD (Hernández-Zimbrón 2018). It is a recombinant humanized Immunoglobulin (Ig) G1 kappa isotype monoclonal antigen-binding fragment (Fab) that targets and binds VEGF-A with high affinity. Aflibercept (Regeneron, Tarrytown, NY; commercialized worldwide by Bayer AG) was approved by the FDA in 2011. It is a fusion protein that combines two key binding domains of human VEGF receptors 1 and 2 and a fragment crystallizable (Fc) region of a human IgG1. Finally, bevacizumab (Genentech Inc., South San Francisco, CA; commercialized worldwide by Roche) is a full-length humanized antibody that binds and blocks all VEGF isoforms.

Oxidative stress can trigger apoptosis, which may activate and recruit macrophages and induce inflammation. Apoptosis may be one of the triggers of the choroidal inflammation and consequently angiogenesis in CNV model (Du 2013).

AIMP2-DX2 is an alternative, antagonistic splicing variant of AIMP2, which is a multifactorial apoptotic gene. AIMP2-DX2 is known to suppress cell apoptosis by hindering the functions of AIMP2. AIMP2-DX2, acting as competitive inhibitor of AIMP2, suppresses TNF-alpha mediated apoptosis through inhibition of ubiquitination/degradation of TRAF2. In addition, it had been reported that AIMP2-DX2 has been confirmed as an existing lung cancer induction factor and, in the existing research, it was confirmed that AIMP2-DX2, manifested extensively in cancer cells, induces cancer by interfering with the cancer suppression functions of AIMP2. Moreover, it was discovered that manifestation of AIMP2-DX2 in normal cell progresses cancerization of cells while suppression of manifestation of AIMP2-DX2, suppresses cancer growth, thereby displaying treatment effects.

It has also been determined that AIMP2-DX2 can be useful in treating neuronal diseases (KR10-2015-0140723 (2017) and US2019/0298858 (2019).

### SUMMARY OF THE INVENTION

Disclosed herein are methods of treating age-related macular disease (AMD) in a subject in need thereof, comprising administering to the subject a pharmaceutically effective amount of a recombinant vector comprising an exon 2-deleted AIMP2 variant (AIMP2-DX2) gene. In some embodiments, the AMD is wet AMD. In some embodiments, the AMD is dry AMD.

The recombinant vector can further comprise an miR-142 target sequence.

The vector can further comprise a promoter operably linked to the AIMP2-DX2. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter or opsin promoter.

The miR-142 target sequence can be 3′ to the AIMP2-DX2 gene.

In some embodiments, the AIMP2-DX2 gene comprises a nucleotide sequence encoding an amino acid sequence that is at least 90% identical to SEQ ID NO:2, 13, 14, 15, 16, 17, 18, 19, or 20.

In some embodiments, the AIMP2-DX2 gene comprises a nucleotide sequence encoding an amino acid sequence of SEQ ID NO:2, 13, 14, 15, 16, 17, 18, 19, or 20.

In some embodiments, the AIMP2-DX2 gene does not have an exon comprising a nucleotide sequence encoding an amino acid sequence that is at least 90% identical to SEQ ID NO:10 or 11.

In some embodiments, the AIMP2-DX2 gene does not have an exon comprising a nucleotide sequence encoding an amino acid sequence of SEQ ID NO:10 or 11.

The miR-142 target sequence can comprise a nucleotide sequence comprising ACACTA. In some embodiments, the miR-142 target sequence comprises ACACTA and 1-17 additional contiguous nucleotides of SEQ ID NO:5. In some embodiments, the miR-142 target sequence comprises a nucleotide sequence at least 50% identical to a nucleotide sequence of SEQ ID NO:5 (TCCATAAAGTAGGAAACACTACA). In some embodiments, the miR-142 target sequence can comprise a nucleotide sequence of SEQ ID NO:5.

In some embodiments, the miR-142 target sequence comprises ACTTTA. In some embodiments, the miR-142 target sequence comprises ACTTTA and 1-15 additional contiguous nucleotides of SEQ ID NO:7. In some embodiments, the miR-142 target sequence comprises a nucleotide sequence at least 50% identical to a nucleotide sequence of SEQ ID NO:7 (AGTAGTGCTTTCTACTTTATG). In some embodiments, the miR-142 target sequence comprises a nucleotide sequence of SEQ ID NO:7.

The miR-142 target sequence can be repeated 2-10 times in the vector disclosed herein.

The vector can be a viral vector. The viral vector can be an adenovirus, adeno-associated virus, lentivirus, retrovirus, human immunodeficiency virus (HIV), murine leukemia virus (MLU), avian sarcoma/leukosis (ASLV), spleen necrosis virus (SNV), Rous sarcoma virus (RSV), mouse mammary tumor virus (MMTV), Herpes simplex virus, or vaccinia virus vector.

In some embodiments, the recombinant vector is administered topically to, by intravitreal injection to, by subconjunctival injection to, or into a subretinal space of the subject.

The methods disclosed herein can further comprise administering to the subject an additional therapeutic agent. In some embodiments, the additional therapeutic agent is ranibizumab, aflibercept, or bevacizumab.

### BRIEF DESCRIPTION OF THE FIGURES

FIG. 1 illustrates an example recombinant vector.

FIG. 2 shows nerve cell-specific expression of a recombinant vector under an in vitro environment.

FIG. 3 shows an miR142-3pT (target) sequence with 4 repeats of miR142-3pT (underlined) (SEQ ID NO:6).

FIG. 4A shows a schematic of miR142-3pT with 1×, 2×, and 3× repeats, and mutant. FIG. 4B shows miR142-3p inhibition on DX2 expression with 1×, 2×, and 3× repeats of miR-142-3pT.

FIG. 5 shows that a core binding sequence is important in DX2 inhibition. A vector with Tseq x3 repeats, which showed significant inhibition of DX2 (FIG. 4B), and DX2 construct were used as controls. 100 pmol of miR-142-3p treatment inhibited Tseq x3 vector significantly but DX2 and mutant sequence were not inhibited.

FIGS. 6A-6C. A comparison of the amino acid sequences of AIMP2-DX2 and variants (FIGS. 6B and 6C are continuations of FIG. 6A).

FIG. 7. Preventive potency of DX2 in the CNV mouse model. Fluorescein angiography and indocyanine green angiography images of laser photocoagulation sites after treatment with scAAV2-GFP or scAAV2-DX2. Twenty-one days after virus injection laser-induced CNV.

FIG. 8. Comparison of choroidal neovascularization area. The ratios of leaky area to CNV area when treated with GFP control and DX2. The leaky area was estimated by measuring the total hyperfluorescent area using FA, and the CNV area was calculated using ICGA. Change in CNV area by treatment with DX2. The CNV volume was calculated based on the total measured Isolectin B4 volume. For each group, n=12, *P<(0.05).

FIG. 9. Expression of VEGF in laser-induced choroidal neovascularization(CNV) mouse model. Western blot result of VEGF expression and VEGF fold change in CNV mouse model. VEGF fold change was measure by Image J. For each group, n=6, *P<(0.05).

FIG. 10. Cross-sectional histology (H&E staining) of retina.

FIGS. 11A-11E. Histological measurements of histological retinal thickness. FIG. 11A. Retina thickness. FIG. 11B. RPE (Retinal Pigment Epithelial) thickness. FIG. 11C. ONL (Outer Nuclear Layer of Photoreceptors) thickness. FIG. 11D. Outer Segment thickness. FIG. 11E. OPL Outer Plexiform Layer) thickness. All the samples were acquired from the optic nerve containing section with 10 μm thickness. Transfection of DX2 gene in retina resulted in the recovery of the total neural retina thickness (FIG. 11A). Transfection of DX2 showed thicker RPE layer (FIG. 11B) and photoreceptor outer segment layer (FIG. 11C), indicating that DX2 expression prevents RPE degeneration and photoreceptor cilia degradation. Transfection of DX2 also showed thicker Outer Nuclear Layer of photoreceptors (FIG. 11D) and Outer Plexiform Layer (FIG. 11E), indicating that DX2 expression reduces photoreceptor degeneration.

FIG. 12. Integrity and proliferation of RPE (Retinal Pigment Epithelial). Transfection of DX2 gene resulted in the recovery of RPE integrity by activating proliferation of RPE.

FIG. 13. PR (Photoreceptor) recovery. Transfection of DX2 gene resulted in recovery of PR population by activating proliferation of PR.

FIGS. 14A-14B. Cellular proliferation of RPE and PR. Ki67 expression was measured to analyze proliferation in RPE and photoreceptor layers. Proliferation in RPE (FIG. 14A) and photoreceptor outer segment layer (FIG. 14B) was significantly higher in the AAV2-DX2 transfected sample.

FIGS. 15A-15F. Functional recovery of retina. Electroretinograph of AAV2-DX2 transfected sample showed increased regaining of normal ERG graph format (FIGS. 15A and 15B). AAV2-DX2 transfection showed increased a-wave amplitude (FIG. 15C) and reduced latency (FIG. 15D) compared to the dry-AMD model (mdm1−/−) or the negative control (mdm1−/−+AAV-GFP), indicating that DX2 expression reduced the damage of photoreceptors' electrophysiological function and visual acuity. AAV2-DX2 transfected sample did not change b-wave amplitude (FIG. 15E) and latency (FIG. 15F).

### DETAILED DESCRIPTION OF THE INVENTION

AIMP2-DX2 is an alternative, antagonistic splicing variant of AIMP2 (aminoacyl tRNA synthase complex-interacting multifunctional protein 2), which is a multifactorial apoptotic gene. AIMP2-DX2 is known to suppress cell apoptosis by hindering the functions of AIMP2.

AIMP2-DX2, acting as a competitive inhibitor of AIMP2, suppresses TNF-alpha mediated apoptosis through inhibition of ubiquitination/degradation of TRAF2.

It has also been determined that AIMP2-DX2 can treat neuronal diseases (US2019/0298858 A1).

It has been also determined that when AIMP2-DX2 is inserted into an adeno-associated virus and the resultant is introduced into subretinal space, it effectively inhibits choroidal neovascularization.

Disclosed herein are methods of treating age-related macular disease (AMD) in a subject in need thereof, comprising administering to the subject a pharmaceutically effective amount of a recombinant vector comprising an exon 2-deleted AIMP2 variant (AIMP2-DX2) gene. In some embodiments, the AMD is wet AMD. In some embodiments, the AMD is dry AMD.

Disclosed herein are methods of decreasing vascular leakage, reducing choroidal neovascularization area, reducing choroidal neovascularization formation, or reducing VEGF expression in the eye or area surrounding the eye in a subject suffering from AMD, comprising administering to the subject a pharmaceutically effective amount of a recombinant vector comprising an exon 2-deleted AIMP2 variant (AIMP2-DX2) gene.

The recombinant vectors as disclosed herein can further comprise an miR-142 target sequence. The vector can further comprise a promoter operably linked to the AIMP2-DX2. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

In the methods disclosed herein, in some embodiments, the recombinant vectors can comprise exon 2-deleted AIMP2 variant (AIMP2-DX2) gene and an miR-142 target sequence. The miR-142 target sequence can be 3′ to the AIMP2-DX2 gene. The vectors described herein can express AIMP2-DX2 in neuronal cells but not in hematopoietic cells, such as leukocytes and lymphoid cells.

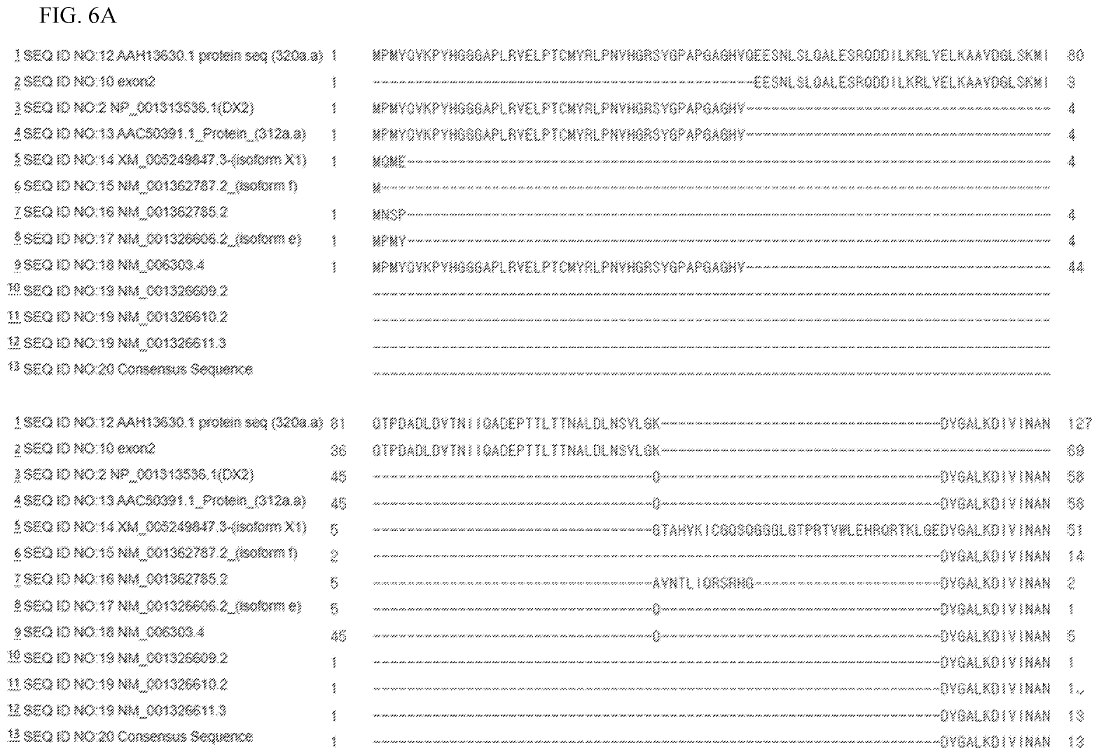

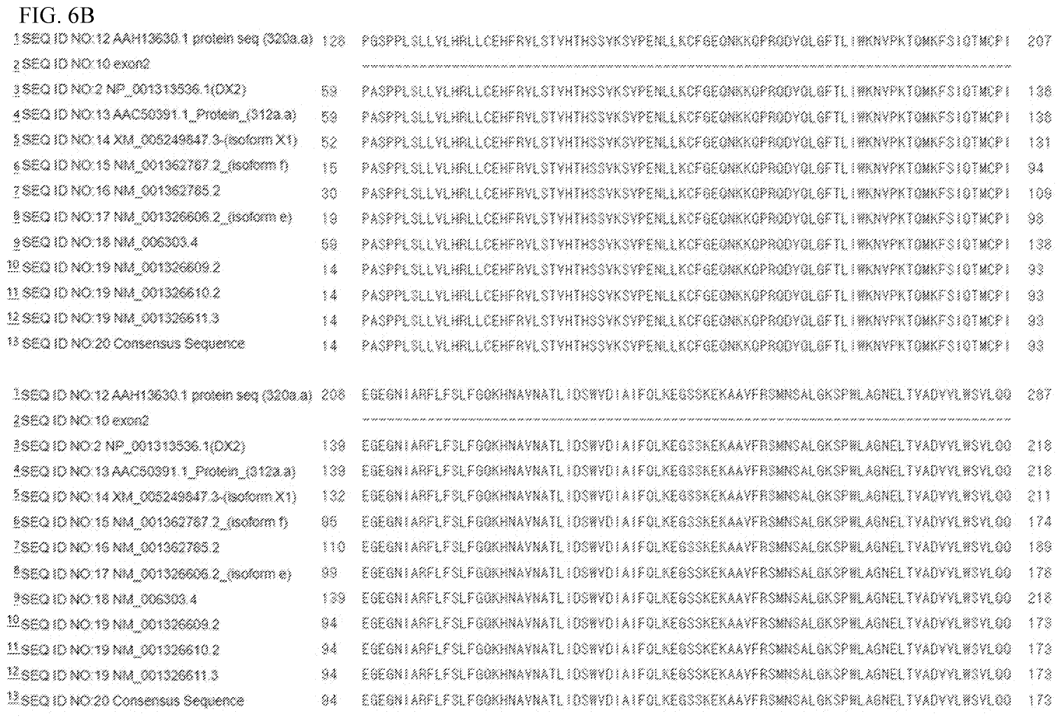

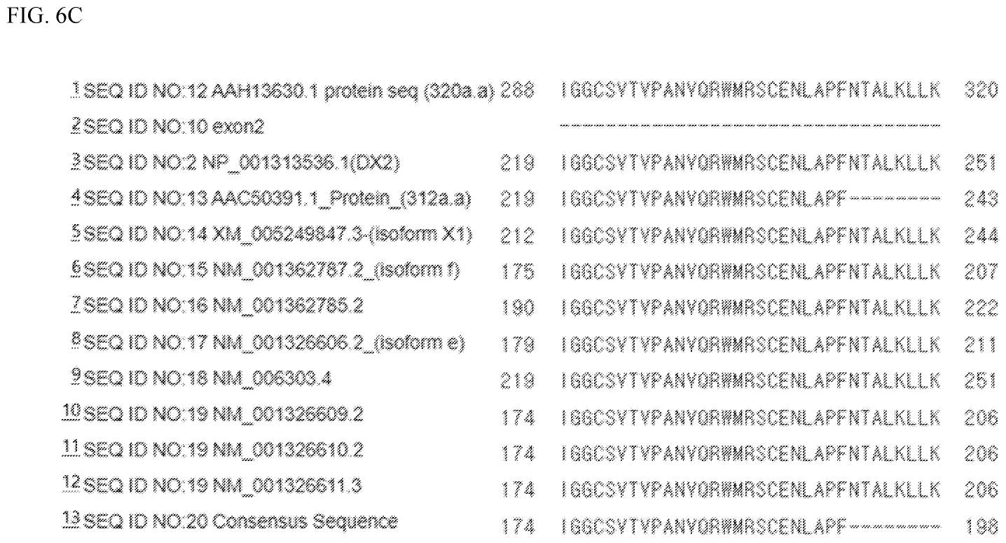

The AIMP2-DX2 polypeptide (SEQ ID NO:2) is a splice variant of AIMP2 (e.g., aa sequence of SEQ ID NO:12; e,g., nt sequence of SEQ ID NO:3), in which the second exon (SEQ ID NO:10; nt sequence of SEQ ID NO:4) of AIMP2 is omitted. In some embodiments, the AIMP2-DX2 gene has a nucleotide sequence set forth in SEQ ID NO:1, and the AI1VIP2-DX2 polypeptide has an amino acid sequence set forth in SEQ ID NO:2. Variants or isoforms of the AIMP2-DX2 polypeptide are also known and can be determined by those in the art (see, e.g., SEQ ID NOS:13-19. For example, FIGS. 6A-6C show a comparison of AIMP2 (SEQ ID NO:2) and variants, SEQ ID NOS:13-19, as well as a consensus or core sequence of AIMP2 or AIMP2-DX2 (SEQ ID NO:20).

In some embodiments, the AIMP2-DX2 gene can comprise a nucleotide sequence encoding an amino acid sequence that is at least 90% identical, at least 93% identical, at least 95% identical, at least 96% identical, at least 97% identical, at least 98% identical, at least 99% identical to SEQ ID NO:2, 13, 14, 15, 16, 17, 18, 19, or 20, or any ranges of % identity therein. The AIMP2-DX2 gene can comprise a nucleotide sequence encoding an amino acid sequence of SEQ ID NO:2, 13, 14, 15, 16, 17, 18, 19, or 20.

The AIMP2-DX2 gene can comprise a nucleotide sequence at least 90% identical, at least 93% identical, at least 95% identical, at least 96% identical, at least 97% identical, at least 98% identical, at least 99% identical to a nucleotide sequence of SEQ ID NO:1, or any ranges of % identity therein. The AIMP2-DX2 gene can comprise a nucleotide sequence of SEQ ID NO:1.

In some embodiments, the AIMP2-DX2 gene does not have an exon comprising a nucleotide sequence encoding an amino acid sequence having at least 70%, at least 75%, at least 80%, at least 85%, at least 90%, at least 95%, at least 96%, at least 97%, at least 98%, at least 99%, or 100% identity to SEQ ID NO:10 or 11. In some embodiments, the AIMP2-DX2 gene does not have an exon comprising a nucleotide sequence encoding an amino acid sequence of SEQ ID NO:10 or 11. In some embodiments, the AIMP2-DX2 gene does not have an exon comprising a nucleotide sequence having at least 70%, at least 75%, at least 80%, at least 85%, at least 90%, at least 95%, at least 96%, at least 97%, at least 98%, at least 99%, or 100% identity to SEQ ID NO:4.

The miR-142 target sequence (miR-142T) can comprise a nucleotide sequence comprising ACACTA. The miR-142 target sequence can comprise a nucleotide sequence comprising ACACTA and 1-17 additional contiguous nucleotides of SEQ ID NO:5. For example, the miR-142 target sequence can comprise a nucleotide sequence comprising ACACTA and a sum of 1, 2, 3, 4, 5, 6, 7, 8, 9, 10, 11, 12, 13, 14, 15, 16, or 17 additional nucleotides that are contiguous 5′ or 3′ of ACACTA as shown in SEQ ID NO:5.

The miR-142 target sequence can comprise a nucleotide sequence at least 50% identical, at least 60% identical, at least 70% identical, at least 80% identical, at least 90% identical, at least 90% identical, at least 93% identical, at least 95% identical, at least 96% identical, at least 97% identical, at least 98% identical, at least 99% identical, or 100% identical to a nucleotide sequence of SEQ ID NO:5 (TCCATAAAGTAGGAAACACTACA; miR-142-3pT). The miR-142 target sequence can comprise a nucleotide sequence of SEQ ID NO:5.

The miR-142 target sequence can comprise a nucleotide sequence comprising ACTTTA. The miR-142 target sequence can comprise a nucleotide sequence comprising ACTTTA and 1-additional contiguous nucleotides of SEQ ID NO:7. For example, the miR-142 target sequence can comprise a nucleotide sequence comprising ACTTTA and a sum of 1, 2, 3, 4, 5, 6, 7, 8, 9, 10, 11, 12, 13, 14, or 15 additional nucleotides that are contiguous 5′ or 3′ of ACTTTA as shown in SEQ ID NO:7.

The miR-142 target sequence can comprise a nucleotide sequence at least 50% identical, at least 60% identical, at least 70% identical, at least 80% identical, at least 90% identical, at least 90% identical, at least 93% identical, at least 95% identical, at least 96% identical, at least 97% identical, at least 98% identical, at least 99% identical, or 100% identical to a nucleotide sequence of SEQ ID NO:7 (AGTAGTGCTTTCTACTTTATG; miR-142-5pT). The miR-142 target sequence can comprise a nucleotide sequence of SEQ ID NO:7.

An example miR142-3pT mutant sequence is:

(SEQ ID NO: 25)
Ccgctgcagtgtgacagtgccagccaatgtgcagaggtggatgaggtct
tgtgaaaacctggctccttttaacacggccctcaagctccttaagtgac
cagaagcttgctagctccataaagtaggaCCACTGCAatcactccataa
agtaggaCCACTGCAagatatctccataaagtaggaCCACTGCAatcac
tccataaagtaggaCCACTGCAaaagcttgtagggatccgcc.

A mutant sequence refers to one or more regions, e.g., four regions, of core sequences of miR142 3pT that are substituted as follows: (5′-AACACTAC-3′→5′-CCACTGCA-3′). Inhibition of DX2 expression in vector transfected HEK293 cells was observed with the miR142-3p x1 repeat (100 pmol) miR142-3p target sequence and as the number of core binding sequence in miR142-3p target seq are increased, miR142-3p inhibition on DX2 expression was also increased. The Tseq x3 core sequence containing vector showed significant inhibition, whereas no inhibition was observed for the mutated 3× sequence.

A microRNA (miRNA) is a non-coding RNA molecule that functions to control gene expression. MiRNAs function via base-pairing with complementary sequences within mRNA molecules, i.e., a miRNA target sequences. miRNAs can bind to target messenger RNA (mRNA) transcripts of protein-coding genes and negatively control their translation or cause mRNA degradation. At present, more than 2000 human miRNAs have been identified and miRbase databases are publicly available. Many miRNAs are expressed in a tissue-specific manner and have an important roles in maintaining tissue-specific functions and differentiation.

MiRNA acts at the post-transcription stage of the gene and, in the case of mammals, and it is known that approximately 60% of the gene expression is controlled by miRNA. miRNA plays an important role in a diverse range of processes within living body and has been disclosed to have correlation with cancer, cardiac disorders and nerve related disorders. For example, miR-142-3p and miR-142-5p exist in miR-142 and any of the target sequences thereof can be used. Thus, “miR-142” or “miRNA-142” refers to, e.g., miR-142-3p and/or miR-142-5p, and can bind to the miR-142 target sequence, e.g., miR-142-3pT or miR-142-5pT.

The miR-142 target sequence can be 5′ or 3′ to the AIMP2-DX2 gene.

For example, “miR-142-3p” can exist in the area at which translocation of its gene occurs in aggressive B cell leukemia and is known to express in hemopoietic tissues (bone marrow, spleen and thymus, etc.). In addition, miR-142-3p is known to be involved in the differentiation of hemopoietic system with confirmation of expression in the liver of fetal mouse (hemopoietic tissue of mouse).

In some embodiments, the miR-142-3p and/or miR-142-5p target sequence is repeated at least 2-10 times, at least 2-8 times, at least 2-6 times, at least 4 times, or any range or number of times thereof.

As an example, the miR-142-3p, e.g., having a nucleotide sequence of SEQ ID NO:23, can have a corresponding target sequence, e.g., a miR-142-3p target sequence (miR-142-3pT) having a nucleotide sequence of SEQ ID NO:5 but not limited thereto. The miR-142-5p, e.g., having a nucleotide sequence of SEQ ID NO:24 can have a corresponding target sequence, e.g., a miR-142-5p target sequence (miR-142-5pT) having a nucleotide sequence of SEQ ID NO:7 but not limited thereto.

In some embodiments, an miR-142-3p can have a nucleotide sequence of SEQ ID NO:23 and an miR-142-5p can have a nucleotide sequence of SEQ ID NO:24.

Disclosed herein are recombinant vectors that can control the side effect of over-expression of the AIMP2-DX2 variant by inserting an miR-142-3p target sequence and/or miR-142-5p target sequence (miR-142-3pT and/or miR-142-5pT, respectively) into a terminal end of AIMP2-DX2, and controlling suppression of AIMP2-DX2 expression in CD45-derived cells, in particular, the lymphatic system and leukocytes. Thus, the expression of AIMP2-DX2 variant can be restricted to only in the injected neuronal cells and tissues but not in non-neuronal hematopoietic cells, the major population in the injected tissue areas. MiR142-3p is expressed only in hematopoietic cells.

Disclosed herein are recombinant vectors containing a target sequence for miR-142-3p and/or miR-142-5p. Disclosed herein are recombinant vectors comprising an exon 2-deleted AIMP2 variant (AIMP2-DX2) gene and miR-142-3p and/or miR-142-5p target sequences as disclosed herein.

The term “recombinant vector” refers to vector that can be expressed as the target protein or RNA in appropriate host cells, and gene construct that contains essential operably linked control factor to enable the inserted gene to be expressed appropriately.

The term “operably linked” refers to functional linkage between the nucleic acid expression control sequence and nucleic acid sequence that codes the targeted protein and RNA to execute general functions. For example, it can affect the expression of nucleic acid sequence that codes promoter and protein or RNA that has been linked for operability of the nucleic acid sequence. Operable linkage with recombinant vector can be manufactured by using gene recombinant technology, which is known well in the corresponding technology area, and uses generally known enzymes in the corresponding technology area for the area-specific DNA cutting and linkage.

The recombinant vectors can further comprise a promoter operably linked to a AIMP2-DX2 as disclosed herein. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

The recombinant vector can additionally contain heterogeneous promoter and operably linked heterogeneous gene in the promoter.

“Heterogeneous gene” as used herein can include protein or polypeptide with biologically appropriate activation, and encrypted sequence of the targeted product such as immunogen or antigenic protein or polypeptide, or treatment activation protein or polypeptide.

Polypeptides can supplement deficiency or absent expression of endogenous protein in host cells. The gene sequence can be induced from a diverse range of suppliers including DNA, cDNA, synthesized DNA, RNA or its combinations. The gene sequence can include genome DNA that contains or does not contain natural intron. In addition, the genome DNA can be acquired along with promoter sequence or polyadenylated sequence. Genome DNA or cDNA can be acquired in various methods. genome DNA can be extracted and purified from appropriate cells through method publicly notified in the corresponding area. Alternatively, mRNA can be used to produce cDNA by reverse transcription or other method by being separated from the cells. Alternatively, polynucleotide sequence can contain sequence that is complementary to RNA sequence, e.g., antisense RNA sequence, and the antisense RNA can be administered to individual to suppress expression of complementary polynucleotide in the cells of individuals.

For example, the heterogeneous gene is an AIMP-2 splicing variant with the loss of exon 2 and miR-142-3p target sequence can be linked to 3′ UTR of the heterogeneous gene. The sequence of the AIMP2 protein (312aa version: AAC50391.1 or GI: 1215669; 320aa version: AAH13630.1, GI: 15489023, BC0 13630.1) are described in the literature (312aa version: Nicolaides, N. C., Kinzler, K. W. and Vogelstein, B. Analysis of the 5′ region of PMS2 reveals heterogeneous transcripts and a novel overlapping gene, Genomics 29 (2), 329-334 (1995)/320 aa version: Generation and initial analysis of more than 15, 000 full-length human and mouse cDNA sequences, Proc. Natl. Acad. Sci. U.S.A. 99 (26), 16899-16903 (2002)).

The term “AIMP2 splicing variant” refers to the variant generated due to partial or total loss of exon 2 among exons 1 to 4. As such, the variant signifies interference of the normal function of AIMP2 by forming AIMP2 protein and heterodimer. The injected AIMP2-DX2 gene is rarely expressed in tissues other than the injected tissue. However, as an additional safety measure, an miR142 target sequence can be inserted to completely block the possibility of AIMP2-DX2 being expressed in hematopoietic cells, the major population of non-neuronal cells in the injected tissue area.

The recombinant vector can include SEQ ID NOS:1 and 5.

The term “% of sequence homology,” “% identity,” or “% identical” to a nucleotide or amino acid sequence can be, e.g., confirmed by comparing the 2 optimally arranged sequence with the comparison domain and some of the nucleotide sequences in the comparison domain can include addition or deletion (that is, gap) in comparison to the reference sequence on the optimal arrange of the 2 sequences (does not include addition or deletion).

Proteins as disclosed herein not only include those with its natural type amino acid sequence but also those with variant amino acid sequences.

Variants of the protein signifies proteins with difference sequences due to the deletion, insertion, non-conservative or conservative substitution or their combinations of the natural amino acid sequence and more than 1 amino acid residue. Amino acid exchange in protein and peptide that does not modify the activation of the molecule in overall is notified in the corresponding area (H. Neurath, R. L. Hill, The Proteins, Academic Press, New York, 1979).

The protein or its variant can be manufactured through natural extraction, synthesis (Merrifield, J. Amer. Chem. Soc. 85: 2149-2156, 1963), or genetic recombination on the basis of the DNA sequence (Sambrook et al, Molecular Cloning, Cold Spring Harbour Laboratory Press, New York, USA, 2^{nd} Ed., 1989).

Amino acid mutations can occur on the basis of the relative similarity of the amino acid side chain substituent such as hydrophilicity, hydrophobicity, electric charge and size, etc. In accordance with the analysis of the size, shape and types of amino acid side chain substituent, it can be discerned that arginine, lysine and histidine are residues with positive charge; alanine, glycine and serine have similar sizes; phenylalanine, tryptophan and tyrosine have similar shapes. Therefore, on the basis of such considerations, arginine, lysine and histidine; alanine, glycine and serine; and phenylalanine, tryptophan and tyrosine can be deemed functional equivalents biologically.

In introducing one or more mutations, hydrophobic index of amino acid can be considered. Hydrophobic index is assigned to each amino acid according to hydrophobicity and charge: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5)

In assigning interactive biological function of protein, hydrophobic amino acid index is very important. It is possible to have similar biological activation only if a substitution is made with an amino acid with a similar hydrophobic index. In the event of introducing a mutation by making reference to the hydrophobic index, substitution between amino acids with hydrophobic index differences within ±2, within ±1, or within ±0.5.

Meanwhile, it is also well known that substitution between amino acids with similar hydrophilicity value can induce proteins with equivalent biological activation. As indicated in U.S. Pat. No. 4,554,101, the following hydrophilic values are assigned to each of the amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine(−1.8); isoleucine (−1.8); tyrosine (−2.3); phenylalanine (−2.5); tryptophan (−3.4).

In the event of introducing one or more mutations by making reference to hydrophilic values, substitutions can be made between amino acids with hydrophilic value differences within ±2, within ±1, or within ±0.5. but not limited thereto.

Amino acid exchange in protein that does not modify the activation of molecule in overall is notified in the corresponding area (H. Neurath, R. L. Hill, The Proteins, Academic Press, New York, 1979). The most generally occurring exchanges are those between the amino acid residues including Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu and Asp/Gly. Vector system can be constructed through diverse methods announced in the corresponding industry. The specific methods are described in Sambrook et al.(2001), Molecular Cloning, A Laboratory Manual, Cold Spring Harbor Laboratory Press.

Vectors disclosed herein can be constructed as a typical vector for cloning or for expression. In addition, the vectors can be constructed with prokaryotic or eukaryotic cells as the host. If the vector is an expression vector and prokaryotic cell is used as the host, it is general to include powerful promoter for execution of transcription (e.g., tac promoter, lac promoter, lacUV5 promoter, 1pp promoter, pL X promoter, pRX promoter, rac5 promoter, amp promoter, recA promoter, SP6 promoter, trp promoter and T7 promoter, etc.), ribosome binding site for commencement of decoding and transcription/decoding termination sequence. In the case of using E. coli (e.g., HB101, BL21, DH5a, etc.) as the host cell, promoter and operator site of the tryptophan biosynthesis route of E. coli (Yanofsky, C. (1984), J. Bacteriol., 158: 1018-1024) and left directional promoter of phage X (pLX promoter, Herskowitz, I. and Hagen, D. (1980), Ann. Rev. Genet., 14: 399-445) can be used as the control site.

Meanwhile, vectors that can be used can be more than 1 type, such as a virus vector, linear DNA, or plasmid DNA.

“Virus vector” refers to a virus vector capable of delivering gene or genetic substance to the desired cells, tissue and/or organ.

Although the virus vectors can include more than 1 species from the group composed of Adenovirus, Adeno-associated virus, Lentivirus, Retrovirus, HIV (Human immunodeficiency virus), MLV (Murine leukemia virus), ASLV (Avian sarcoma/leukosis), SNV (Spleen necrosis virus), RSV (Rous sarcoma virus), MMTV (Mouse mammary tumor virus) and Herpes simplex virus, it is not limited thereto. In some embodiments, the viral vector can be an adeno-associated virus (AAV), adeonovirus, lentivirus, retrovirus, vaccinia virus, or herpes simplex virus vector.

Although Retrovirus has an integration function for the genome of host cells and is harmless to the human body, it can have characteristic including suppressing functions of normal cells at the time of integration, ability to infect a diverse range of cells, ease of proliferation, accommodate approximately 1-7 kb of external gene, and generate duplication deficient virus. However, Retroviruses can also have disadvantages including difficulties in infecting cells after mitotic division, gene delivery under an in vivo condition, and need to proliferate somatic cells under in vitro condition. In addition, Retroviruses have the risk of spontaneous mutations as it can be integrated into proto-oncogene, thereby presenting the possibility of cell necrosis.

Meanwhile, Adenoviruses have various advantages as a cloning vector including duplication even in nucleus of cells in medium level size, clinically nontoxic, stable even if external gene is inserted, no rearrangement or loss of genes, transformation of eukaryotic organism and stably undergoes expression at high level even when integrated into host cell chromosome. Good host cells of Adenoviruses are the cells that are the causes of hemopoietic, lymphatic and myeloma in human. However, proliferation is difficult since it is a linear DNA and it is not easy to recover the infected virus along with low infection rate of virus. In addition, expression of the delivered gene is most extensive during 1-2 weeks with expression sustained over the 3-4 weeks only in some of the cells. Another issue is that it has high immuno-antigenicity.

Adeno-associated virus (AAV) has been preferred in recent years since it can supplement the aforementioned problems and has a lot of advantages as gene therapy agent. It is also referred as adenosatellite virus. Diameter of adeno-associated virus particle is 20 nm and is known to have almost no harm to human body. As such, its sales as gene therapy agent in Europe were approved.

AAV is a provirus with single strand that needs auxiliary virus for duplication and AAV genome has 4,680 bp that can be inserted into specific area of the chromosome 19 of the infected cells. Trans-gene is inserted into the plasma DNA connected by the 2 inverted terminal repeat (ITR) sequence section with 145 bp each and signal sequence section. Transfection is executed along with other plasmid DNA that expresses the AAV rep and cap sections, and Adenovirus is added as an auxiliary virus. AAV has the advantages of wide range of host cells that deliver genes, little immunological side effects at the time of repetitive administration and long gene expression period. Moreover, it is safe even if the AAV genome is integrated with the chromosome of host cells and does not modify or rearrange the gene expression of the host.

The Adeno-associated virus is known to have a total of 4 serotypes. Among the serotypes of many Adeno-associated viruses that can be used in the delivery of the target gene, the most widely researched vector is the Adeno-associated virus serotype 2 and is currently used in the delivery of clinical genes of cystic fibrosis, hemophilia and Canavan's disease. In addition, recently, the potential of recombinant adeno-associated virus (rAAV) is increasing in the area of cancer gene therapy (Du 2013). In some embodiments, the Adeno-associated virus serotype 2 can be used. Although it is possible to select and apply appropriate viral vector, it is not limited to this.

In addition, if the vectors are expression vectors and use eukaryotic cells as the host, promoter derived from the genome of mammalian cells (e.g., metallothionein promoter) or promoter derived from mammalian virus (e.g., post-adenovirus promoter, vaccine virus 7.5K promoter, SV40 promoter, cytomegalovirus promoter and HSV TK promoter) can be used. Specifically, although it can include more than 1 species selected from the group composed of promoters selected from the group composed of LTR of Retrovirus, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter and opsin promoter, it is not limited to these. Moreover, it generally has polyadenylated sequence as the transcription termination sequence.

Vectors disclosed herein can be fused with other sequences as need to make the purification of the protein easier. Although the fused sequence such as glutathione S-transferase (Pharmacia, USA), maltose binding protein (NEB, USA), FLAG (IBL USA) and 6×His (hexahistidine; Quiagen, USA), etc. can be used, e.g., it is not limited to these. In addition, expression vectors can include tolerance gene against antibiotics generally used in the corresponding industry as the selective marker including Ampicillin, Gentamycin, Carbenicillin, Chloramphenicol, Streptomycin, Kanamycin, Geneticin, Neomycin and Tetracycline, as examples.

In addition, disclosed herein are gene carriers including the recombinant vector containing a target sequence (miR-142-3pT and/or miR-142-5pT) for miR-142, such as miR-142-3p and/or miR-142-5p, respectively.

The term “gene transfer” includes delivery of genetic substances to cells for transcription and expression in general. Its method is ideal for protein expression and treatment purposes. A diverse range of delivery methods such as DNA transfection and virus transduction are announced. It signifies virus-mediated gene transfer due to the possibility of targeting specific receptor and/or cell types through high delivery efficiency and high level of expression of delivered genes, and, if necessary, nature-friendliness or pseudo-typing.

The gene carriers can be transformed entity that has been transformed into the recombinant vector, and transformation includes all methods of introducing nucleic acid to organic entity, cells, tissues or organs, and, as announced in the corresponding area, it is possible to select and execute appropriate standard technology in accordance with the host cells. Although such methods include electroporation, fusion of protoplasm, calcium phosphate (CaPO4) sedimentation, calcium chloride (CaC12) sedimentation, mixing with the use of silicone carbide fiber, agribacteria-mediated transformation, PEG, dextran sulphate and lipofectamin, etc., it is not limited to these.

The gene carriers are for the purpose of expression of heterogeneous genes in neuron. As such it suppresses the expression of the heterogeneous gene in CD45-derived cells and can increase the expression of heterogeneous gene in brain tissue. Majority of the CD45 are transmembrane protein tyrosine phosphatase situated at the hematopoietic cell. Cells can be defined in accordance with the molecules situated on the cell surface and the CD45 is the cell marker for all leukocyte groups and B lymphocytes. The gene carrier is not be expressed in the CD45-derived cells, in particular, in lymphoid and leukocyte range of cells.

The gene carriers can additionally include carrier, excipient or diluent allowed to be used pharmacologically.

In addition, disclosed herein are methods of delivering and expressing the heterogeneous gene in the neuron that includes the stage of introducing the recombinant vector into the corresponding entity.

The methods can increase the expression of heterogeneous gene in cerebral tissues and control heterogeneous gene expression in other tissues.

In addition, disclosed herein are vectors comprising 1) a promoter; 2) a nucleotide sequence that encodes a target protein linked with the promoter to enable operation; and 3) an expression cassette that includes the nucleotide sequence targeting miR-142-3p inserted into 3′UTR of the nucleotide sequence. In some embodiments, the vectors can comprise 1) a promoter; 2) a nucleotide sequence that encodes a target protein linked with the promoter to enable operation; and 3) an expression cassette that includes the nucleotide sequence targeting miR-142-5p inserted into 3′UTR of the nucleotide sequence.

The term “expression cassette” refers to the unit cassette that can execute expression for the production and secretion of the target protein operably linked with the downstream of signal peptide as it includes gene that encodes the target protein and nucelotide sequence that encodes the promoter and signal peptide. Secretion expression cassette can be used mixed with the secretion system. A diverse range of factors that can assist the efficient production of the target protein can be included in and out of such expression cassette.

In addition, disclosed herein are preventive or therapeutic preparations for AMD that includes a nucleotide sequence that encodes AIMP-2 splicing variant with loss of exon 2 and nucleotide sequence that targets miR-142-3p linked to 3′UTR of the nucleotide sequence.

Accordingly, also disclosed herein are methods of treating AMD in a subject in need thereof, comprising administering any of the vectors disclosed herein. In some embodiments, the AMD is wet AMD. In some embodiments, the AMD is dry AMD.

The vectors disclosed herein can effect, but not limited to, apoptosis inhibition, dyskinesia amelioration, and/or oxidative stress inhibition, and thus prevent or treat AMD.

The term “treatment” includes not only complete treatment of AMD but also partial treatment, improvement and/or reduction in the overall symptoms of AMD as results of application of the pharmacological agent disclosed herein.

The term “prevention” signifies prevention of the occurrence of overall symptoms of AMD in advance by suppressing or blocking the symptoms or phenomenon such as cognition disorder, behavior disorder and destruction of brain nerves by applying pharmacological agents disclosed herein to the entity with degenerative cerebral disorders.

Adjuvants other than the active ingredients can be included additionally to the pharmacological agents disclosed herein. Although any adjuvant can be used without restrictions as long as it is known in the corresponding technical area, it is possible to increase immunity by further including complete and incomplete adjuvant of Freund, for example.

Pharmacological agents disclosed herein can be manufactured in the format of having mixed the active ingredients with the pharmacologically allowed carrier. Here, pharmacologically allowed carrier includes carrier, excipient and diluent generally used in the area of pharmacology. Pharmacologically allowed carrier that can be used for the pharmacological agents disclosed herein include lactose, dextrose, sucrose, sorbitol, mannitol, xylitol, erythritol, malitol, starch, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil, but not limited to these.

Pharmacological agents disclosed herein can be used by being manufactured in various formats including oral administration types such as powder, granule, pill, capsule, suspended solution, emulsion, syrup and aerosol, etc., and external application, suppository drug or disinfection injection solution, etc. in accordance with their respective general manufacturing methods.

When manufactured into preparations, diluents or excipients such as filler, extender, binding agent, humectant, disintegrating agent and surfactant, etc., which are used generally, can be used in the manufacturing. Solid preparations for oral administration include pill, tablet, powder, granule and capsule preparations, and such solid preparations can be manufactured by mixing more than 1 excipient such as starch, calcium carbonate, sucrose, lactose and gelatin with the active ingredients. In addition, lubricants such as magnesium stearate and talc can also be used in addition to simple excipients. Liquid preparations for oral administration include suspended solution, solution for internal use, oil and syrup, etc. with the inclusion of various excipients such as humectant, sweetening agent, flavoring and preservative, etc. other than water and liquid paraffin, which are the generally used diluents. Preparations for non-oral administration include sterilized aqueous solution, non-aqueous solvent, suspension agent, oil, freeze dried agent and suppository. Vegetable oil such as propylene glycol, polyethylene glycol and olive oil, and injectable esters such as ethylate can be used as non-aqueous solvent and suspension solution. Agents for suppository can include witepsol, tween 61, cacao oil, laurine oil and glycerogelatin, etc.

Pharmacological agents disclosed herein can be administered into entity through diversified channels. All formats of administration such as oral administration, and intravenous, muscle, subcutaneous and intraperitoneal injection can be used.

In some embodiments, the recombinant vector is administered topically to, by intravitreal injection to, by subconjunctival injection to, or into a subretinal space of the subject.

The methods disclosed herein can further comprise administering to the subject an additional therapeutic agent(s). In some embodiments, the additional therapeutic agent is ranibizumab, aflibercept, and/or bevacizumab.

Desirable doses of administration of therapeutic agents disclosed herein differs depending on various factors including preparation production method, administration format, age, weight and gender of the patient, extent of the symptoms of the disease, food, administration period, administration route, discharge speed and reaction sensitivity, etc. Nonetheless, it can be selected appropriately by the corresponding manufacturer.

However, for the treatment effects, skilled medical doctor can determine and prescribe effective dose for the targeted treatment. For example, the treatment agents include intravenous, subcutaneous and muscle injection, and direction injection into cerebral ventricle or spinal cord by using micro-needle. Multiple injections and repetitive administrations are possible, e.g., the effective dose is 0.05 to 15 mg/kg in the case of vector, 5×10¹¹ to 3.3×10¹⁴ viral particle (2.5×10¹² to 1.5×10¹⁶ IU)/kg in the case of recombinant virus and 5×10² to 5×10⁷ cells/kg in the cells. Desirably, the doses are 0.1 to 10 mg/kg in the case of vector, 5×10¹² to 3.3×10¹³ particles (2.5×10¹³ to 1.5×10¹⁵ IU)/kg in the case of recombinant virus and 5×10³ to 5×10⁶ cells/kg in the case of cells at the rate of 2 to 3 administrations per week. The dose is not strictly restricted. Rather, it can be modified in accordance with the condition of the patient and the extent of manifestation of the neural disorders. Effective dose for other subcutaneous fat and muscle injection, and direct administration into the affected area is 9×10¹⁰ to 3.3×10¹⁴ recombinant viral particles with the interval of 10 cm and at the rate of 2-3 times per week. The dose is not strictly restricted. Rather, it can be modified in accordance with the condition of the patient and the extent of manifestation of the neural disorders. More specifically, pharmacological agents disclosed herein can include 1×10¹⁰ to 1×10¹² vg (virus genome)/mL of recombinant adeno-associated virus and, generally, it is advisable to inject 1×10¹² vg once every 2 days over 2 weeks. It can be administered once a day or by dividing the dose for several administrations throughout the day. In some embodiments, the vectors can be administered in a dose of 0.1×10⁸ vg to 500×10⁸ vg, 1×10⁸ vg to 100×10⁸ vg, 1×10⁸ vg to 10×10⁸ vg, e.g., 5×10⁸ vg, or any ranges derived therefrom. For IV injections, e.g., vg can be translated to doses for human based on body weight for IV injection. For local tissue injections, e.g., vg can also be translated to doses for humans based on the target cell number and effective MOI (multiplicity of infection).

In some embodiments, the vectors disclosed herein can be injected to a subject by, e.g., subretinal injection, intravitreal injection, or subchoroidal injection. The injection can be in the form of a liquid. In other embodiments, the vectors disclosed herein can be administered to a subject in the form of eye drops or ointment.

The pharmacological preparations can be produced in a diverse range of orally and non-orally administrable formats. In some embodiments, the vector disclosed herein can be administered to the brain or spinal cord. In some embodiments, the vectors disclosed herein can be administered to the brain by stereotaxic injection.

Orally administrative agents include pills, tablets, hard and soft capsules, liquid, suspended solution, oils, syrup and granules, etc. These agents can include diluent (e.g., lactose, dextrose, sucrose, mannitol, sorbitol, cellulose and/or glycine) and glidents (e.g., silica, talc, and stearic acid and its magnesium or calcium salts, and/ or polyethylene glycol) in addition to the active ingredients. In addition, the pills can contain binding agents such as magnesium aluminum silicate, starch paste, gelatin, tragacanthin, methyl cellulose, sodium carboxymethyl cellulose and/or polyvinyl pyrrolidine, and, depending on the situation, can contain disintegration agent such as starch, agar, alginic acid or its sodium salt or similar mixture and/or absorbent, coloring, flavor and sweetener. The agents can be manufactured by general mixing, granulation or coating methods.

In addition, injection agents are the representative form of non-orally administered preparations. Solvents for such injection agents include water, Ringer's solution, isotonic physiological saline and suspension. Sterilized fixation oil of the injection agent can be used as solvent or suspension medium, and any non-irritating fixation oil including mono- and di-glyceride can be used for such purpose. In addition, the injection agent can use fatty acids such as oleic acid.

The invention will be explained in more detail by using the following execution examples below. However, the following execution examples are only for the purpose of specifying the contents of the invention and do not limit the application of the invention to such examples.

### EXAMPLES

### Example 1

### Production of the Recombinant Vector

Majority of CD45 are transmembrane protein tyrosine phosphatase of the hematopoietic cell, which can be used to define the cells in accordance with the molecule on the cell surface. CD45 is a marker for all leukocyte groups and B lymphocytes. A recombinant vector has been produced that is expressed specifically and only in neurons without being expressed in CD45-derived cells, in particular, lymphoid and leukocyte cells. The recombinant vector contains a splicing variant in which exon 2 of the Aminoacyl tRNA Synthetase Complex Interacting Multifunctional Protein 2 (AIMP2) has been deleted and an miRNA capable of controlling the expression of the AIMP2 splicing variant.

The recombinant vector was produced as a distribution safety measure in order to induce specific expression of the AIMP2 splicing variant in injected neuronal tissues. Also this was done to completely block any possibility of AIMP2-DX2 being expressed in hematopoietic cells, which is the major population of non-neuronal cells in the injected tissue area.

### Example 1-1

### Production of AIMP2 Variant

AIMP2 is one of the proteins involved in the formation of aminoacyl-tRNA synthetase (ARSs) and acts as a multifactorial apoptotic protein. In order to construct a plasmid that expresses the variant in which exon 2 of the AIMP2 has been deleted, cDNA of AIMP2 splicing variant was cloned into pcDNA3.1-myc. The sub-cloning in pcDNA3.1-myc was executed by using EcoR1 and Xho1 after having amplified the AIMP2 splicing variant by using a primer having EcoR1 and Xho1 linker attached to the H322 cDNA.

AIMP2 variant having a nucleotide sequence of SEQ ID NO:1 and an amino acid sequence of SEQ ID NO:2 was used.

### Example 1-2

### Sorting of miRNA and Selection of its Target Sequence

As mentioned above, as a distribution safety measure, the recombinant vector was produced as above in order to confine the expression of the AIMP2 variant in injected neuronal cells and to completely block the possibility of AIMP2-DX2 being expressed in hematopoietic cells, the major population of non-neuronal cells in the injected tissue area.

For this purpose, miR-142-3p that is specifically expressed only in hematopoietic cells that generate leukocyte and lymphoid related cells was selected as the target. In order to produce the sequence that targets only the miR-142-3p, microarray data of mouse B cells and computer programming of genes targeted by miR⁻142-3p (mirSVR score) were used. The miR-142-3p is a nucleotide sequence indicated SEQ ID NO:. The miR-142-3p target sequence of SEQ ID NO:5 binds to miR-142-3p

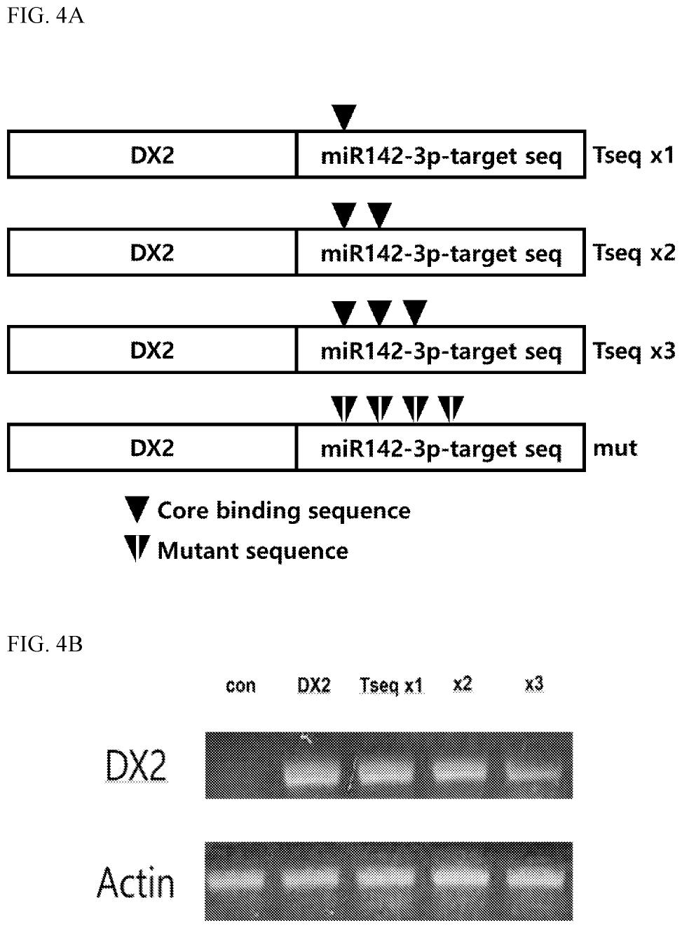

The miR-142-3p target sequence includes Nhe 1 and Hind III, Bmt 1 site sequence (ccagaagcttgctagc; SEQ ID NO:21) and Hind H site sequence (aagcttgtag; SEQ ID NO:22). The miR-142-3pT can comprise the nucleotide sequence of SEQ ID NO:5 that has been repeated 4 times with the linkers (tcac and gatatc) that connects them (FIG. 4; SEQ ID NO:6).

### Example 1-3

### Production of the Recombinant Vector

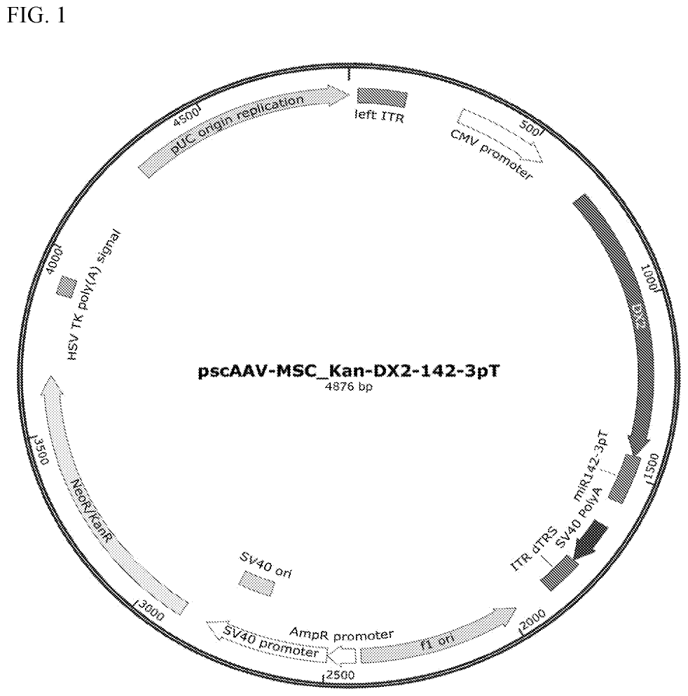

In order to produce the recombinant vector, the miR-142-3p target sequence (SEQ ID NO:5) was inserted into 3′UTR of the AIMP2 variant (sequence number of 1). Connecting of the AIMP-2 variant and miR-142-3p target sequence is indicated with base sequence number of 6, and, specifically, was cut and inserted by using Nhe I and Hind III sites. The recombinant vector is shown in FIG. 1.

### Example 2

### Confirmation of the Nerve Cells Specific Expression of Recombinant Vector in Vitro

Since miR142-3p is specifically expressed only in hemopoietic cells, the extent of the expression of AIMP2 variant was confirmed in specific cells in accordance with the knockdown of AIMP2 variant according to the expression of miR142-3p target sequence of the recombinant vector.

Specifically, there were group with no treatment of the recombinant vector (SHAM), void/control vector processed group (NC vector), single AIMP2 variant vector processed group (pscAAV_DX2) and group treated with the recombinant vector (pscAAV-DX2-miR142-3pT). The concentration of all the vectors is in the unit of ug/ul and each group was treated with 2.5 ul (2.5 ug). In each of the treatment groups, treatments were made on the THP-1 cells strain (human leukemic monocyte cells) and SH-SY5Y cells strain (neuroblastoma) with confirmation of knockdown of AIMP2 variant. qPCR was executed by using the primers in the Table 1 below (degeneration for 15 seconds, and annealing and extension over 40 cycles under the temperature of 60° C. for 30 seconds).

TABLE 1

| AIMP2 | | SEQ |
|---|---|---|
| variant | Primer | ID NO: |
| Forward | CTGGCCACGTGCAGGATTACGGGG | 8 |
| | (only human) | |
| Reverse | AAGTGAATCCCAGCTGATAG | 9 |
| | (only human) | |

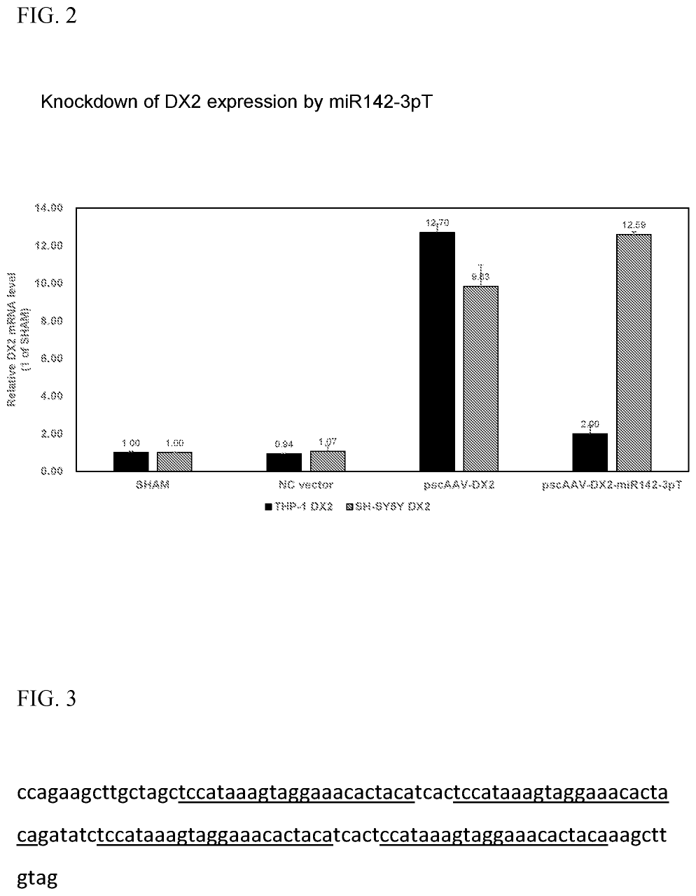

As a result, it was confirmed that AIMP2 variant is not expressed in the SHAM and NC vector groups. In addition, it was confirmed that there was expression in both the THP-1 cell strain and SH-SY5Y cell strain of the single AIMP2 variant vector processed group (pscAAV-DX2), thereby confirming that nerve cell-specific expression is not induced. On the other hand, it was confirmed that the AIMP2 variant is specifically expressed only in the SH-SY5Y cell strain in the group treated with the recombinant vector (FIG. 2).

### Example 3

### Materials and Methods

### Example 3-1

### qRT-PCR

Total RNA was isolated from spinal cord using TRIzol (Invitrogen, Waltham, MA, USA) according to the manufacturer's protocol. The extracted RNA was quantified by a spectrophotometer (ASP-2680, ACTgene, USA) for quantification. For making cDNA, a reverse transcription was performed using the SuperScript III First-Strand (Invitrogen) through manufacturer's protocol. The resulting cDNA was used for real-time PCR using SYBR green PCR master mix (ThermoFisher Scientific, USA). Expression data of the duplicated result were used for 2-AACt statistical analysis and GADPH expression was used for normalization.

### Example 3-2

### miR142-3p Inhibition Experiment

miR-142-3p inhibition on DX2 expression could be observed from x1 miR-142-3p target sequence. The HEK293 cells were transiently transfected with the x1, x2, and x3 repeat miR-142-3p target sequence vectors, and also with 100 pmol miR-142-3p using lipofectamine 2000 (Invitrogen, US), and then incubated for 48 hrs. The amount of DX2 mRNA was analyzed by PCR. miR142-3p inhibition on DX2 expression was observed from Tseq x1 repeat miR142-3p target seq (FIG. 4B).

### Example 4

### Example 4-1

### 3 Types of Vectors Generated for Inhibition Effect of Core Binding Sequence

Tseq x1 contains 1 core binding sequence, Tseq x2 contains 2 core binding sequences, and Tseq x3 contains 3 core binding sequences (FIG. 4A).

miR142-3p (100 pmol) inhibition on DX2 expression was started to be observed from x1 repeat miR142-3p target sequence. The HEK293 cells were transiently transfected with the x1, x2, and x3 repeat miR-142-3p T seq vectors, and also with 100 pmol miR-142-3p using lipofectamin 2000 (invitrogen, US), then incubated for 48 h. Amount of DX2 mRNA was analyzed by PCR. When the number of core binding sequence in miR142-3p target seq are increased, miR142-3p inhibition on DX2 expression was also increased. Tseq x3 core sequence containing vector showed significant inhibition (FIG. 4B).

### Example 4-2

### Core Sequence Mutation

Using mouse B cell microarray data and mirSVR score of miR-142-3p target gene, core sequence was predicted. Four regions of core sequences were substituted as follows: (5′-AACACTAC-3 [symbol] 5′-CCACTGCA-3′) (see FIG. 3 for original sequence and FIG. 4A for schematic drawing).

### Example 4-3

### Core Binding Sequence is Important DX2 Inhibition

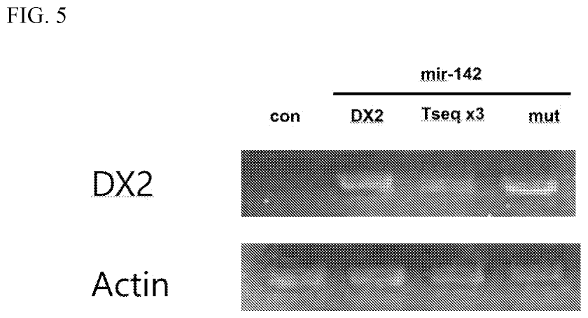

Four core sequences were substituted (FIG. 4A). The HEK293 cells were transiently transfected with the DX2-miR-142-3p T seq x3 repeated vector (Tseq3×) or with core sequence mutated vector (mut), and with 100 pmol miR-142-3p by using lipofectamin 2000 (Invitrogen, US), and then incubated for 48 hrs. Expression of DX2 mRNA was analyzed by PCR. Tseq x3 repeated vector which showed significant inhibition of DX2 (FIG. 4B) and DX2 construct were used as control. 100 pmol of miR142-3p treatment inhibited Tseq x3 vector significantly but DX2 and mut sequence were not inhibited (FIG. 5).

### Example 5

### Wet AMD Mouse Model

### Material and Methods

### 5-1

### Animal Experiments

The animals were housed in individual cages under specific pathogen-free conditions and a constant environment condition (21° C.-23° C. temperature, 50-60% humidity and 12-h light/dark cycle) in the animal facility. The mouse Ocular sinister (OS, left eye) in each group treated AAV-GFP and Ocular Dexter (OD, right eye) treated AAV-DX2. (Injection: 5×10⁸ vg). After sub-retinal injection 1 week and 10 weeks, using laser photo-coagulator, RPE layer of eye fundus induce laser to make 3 weeks-, 3 months-wet AMD model, respectively. After 2 weeks, 2 months of laser treatment, the mouse eye ball is isolated.

### 5-1-1

### Sub-Retinal Injection was Performed Using the Following Protocol

Anesthetize the rodent. Use intraperitoneal injections of 100 mg/ml ketamine and 10 mg/ml xylazine (20 μl/10 g body weight) over isofluorane inhalation. Ensure that the animal is deeply anesthetized by pinching one of its paws. If it flinches, wait several more minutes and try again before beginning the sub-retinal injection. Position the rodent onto its side so that the eye that will be injected is facing the ceiling. Under a dissecting microscope gently stretch the skin so the eye pops slightly up out of the socket (temporary proptosis) and becomes more accessible by holding its head with two fingers just above the ear and by its jaw and gently stretch the skin parallel to the eyelids so that the eye pops slightly up out of the socket. 38G sterile micro-tip needle (INCYTO, KR), make a hole immediately below the limbus and at an angle to avoid touching the lens with the needle. Retract the disposable sharp needle from the eye while maintaining the grip on the head. After either mounting the pre-loaded syringe with a blunt needle on a micromanipulator or holding it by hand, insert the tip of the syringe with the blunt needle through the hole, taking care again not to touch the lens and gently push it through the eye very gently until feeling resistance. Keeping all movements to a minimum, carefully inject the viruses slowly into the sub-retinal space. AAV2-GFP was injected at OS (left). AAV2-DX2 was injected at OD (right). Retract the syringe slowly. Apply eye moisturizing drops to keep the eye hydrated. Continue to monitor the animal until it regains sternal recumbency.

### 5-1-2

### Mouse Laser-Induced CNV Model was Obtained Using the Following Protocol

Before the induced Laser to mice, Position the laser and slit lamp where it can be easily accessible. Turn on laser and set to pre-determined parameters. Anesthetize the rodent. Use intraperitoneal injections of 100 mg/ml ketamine and 10 mg/ml xylazine (20 μl/10 g body weight) over isofluorane inhalation. Roll mouse on its side and place a drop (approximately 30 μl) of tetracaine hydrochloride into each eye for topical anesthesia. Wait 2 min for solution to take effect.

Repeat previous step with one drop of topical tropicamide for pupillary dilation. Alternatively, use phenylephrine hydrochloride (2.5%) for dilation. After appropriate time has elapsed, quickly place the mouse on the mouse stage and place the stage on chin rest of slit lamp. Turn on slit lamp to the lowest light brightness and check the degree of pupillary dilation. If pupil is not adequately dilated (approximately 2.5-3 mm), return mouse to animal warmer and wait. Alternatively, administer another drop of tropicamide. Once eye is sufficiently dilated, proceed to laser procedure.

Adjust the placement of mouse on the mouse-stage, so that it is ideally positioned for visualization of optic nerve. Orient the mouse on its holder so it lies horizontally, perpendicular to slit lamp beam, with the head at one side and tail at the other.

Slightly turn the mouse so it is at an approximately 170° angle with the head closer to laser operator.

After the mouse is ideally positioned, place one drop of artificial tear solution on a 25 mm×25 mm glass coverslip. Place one drop of artificial tear solution on the mouse's opposite eye—this will ensure the eye is hydrated and help delay cataract formation.

Hold corner of coverslip between thumb and pointer fingers; position so that the glass is squeezed between tips of both fingers. Gently wrap the remaining three fingers around the animal's body for support and hand stabilization. Position hand so that the glass coverslip can be easily placed on the mouse's eye. Once stable position is obtained, carefully press glass coverslip (with drop of artificial tear still adhered) onto the mouse's eye. Make sure the coverslip is positioned as perpendicular as possible to the laser beam in order to prevent laser beam scatter or reflection. The coverslip acts as a contact lens to flatten the cornea. Look through slit lamp and with free hand toggle focus until retina can be visualized. The retina will have a light-yellow/red color depending on the location visualized, distinct, red vessels will be visible. Slowly and carefully manipulate mouse head and/or coverslip until visualizing the optic nerve. The optic nerve will be yellow in color with multiple vessels radiating from it. Once operator has confirmed visualization of optic nerve, turn on laser focusing beam. Once laser beam has been turned on, maneuver laser focusing beam to desired position (approximately ldisc diameter from the optic nerve). Focus laser beam on the RPE of the eye fundus. Proper focus is achieved by having the sharpest and clearest laser beam. If aiming beam looks oval or out of focus, toggle slit lamp focus or re-position glass coverslip. Once the aiming beam is focused on RPE, initiate laser administration using the laser's foot trigger. Watch for the appearance of a bubble immediately after laser administration. The outline of the laser shot should be clear and not hazy in any way. Repeat previous 3 steps for all desired CNV positions (usually at 3, 6, 9, and 12 o'clock positions around optic nerve).

Record in a notebook the location and result of each laser shot administration and result (successful, hazy, hemorrhage, etc.) of each administered shot for the eye. Be sure to place the laser in stand-by mode when not in use. Repeat previous all steps for the mouse's other eye, if needed, using the opposite hand for stabilization and a new coverslip. After all desired laser shots are administered, turn off laser and slit-lamp.

Discard coverslip and place mouse on warmer for recovery from anesthesia. acroscopically inspect eye for any injury and place a drop of artificial tear solution to keep the eye hydrated and potentially prevent future cataract development. Once mouse recovers from anesthesia, return to cage.

### 5-1-3

### Fluorescein Angiography

Eyes were fixed in 4% paraformaldehyde (Electron Microscopy Sciences, Hatfield, PA) for 2 hours after removal of the cornea and lens. Posterior eyecups of the RPE/choroid/sclera were dissected, and the vitreous was removed. Eyecups were incubated overnight at 4° C. with (AlexaFluor 647 or FITC)-conjugated Isolectin B4 (1:200, Invitrogen, Carlsbad, CA) to label invading choroidal vessels.

### 5-1-4

### Expression of Angiogenic Factor

Cells were collected and lysed with PBS containing 1% Triton X-100. Equal amounts of proteins were loaded into the wells of the SDS-PAGE gel and transferred to nitrocellulose membrane filters for 2 hrs at 100V. The membranes were blocked with PBST (phosphate buffered saline with Tween 20) containing 1% BSA for 1 hr at room temperature, and probed with anti-VEGF and anti-beta actin antibodies for 1 hr at room temperature. The membranes were washed three times with PBST, followed by incubation with a secondary antibody for 1 hr at room temperature. Following three washings with PBST, immune-reactive band were detected. The data were quantified using Image J software.

### Example 5-2

### Results

scAAV2-DX2 treated mice attenuates laser-induced choroidal neovascularization.

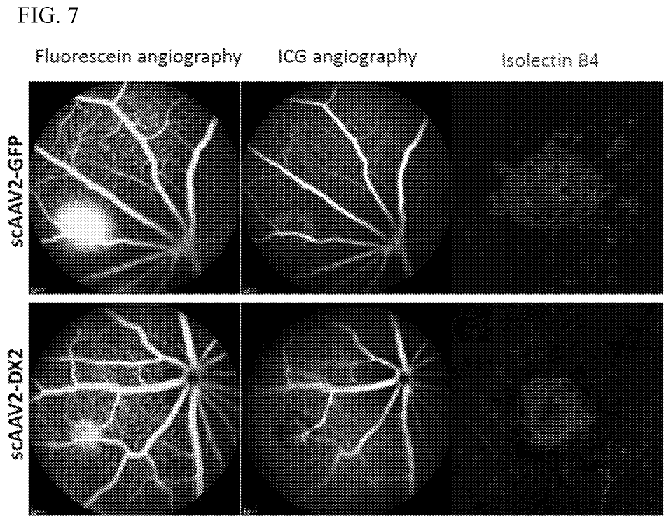

Laser-induced choroidal neovascularization (CNV) is widely used animal model for wet AMD. In this model, laser is used to disrupt Bruch's membrane, which allows the underlying choroidal vessels to penetrate and grow into the space underneath the pigment epithelium. Subretinal injection of scAAV2-GFP (control) or scAAV2-DX2 to 5-wk-old male C57BL/6 mice (n=12) were conducted (Day 0). Twenty-one days after injection, CNV was produced by laser photocoagulation (Day 21). Fourteen days after laser treatment, Fluorescein angiography and ICG angiography performed (Day 35). Next day, mice were killed and choroidal flat mounts generated and stained with (FITC)-conjugated isolectin B4 (FIG. 7) (Day 36). Vascular leakage caused by the new blood vessel formation was clearly observed at the laser-induced photocoagulation site by fluorescein angiography. ICG angiography is used to acquire an angiogram of the choroid. Flat mounts were used to evaluated the presence and area of clearly demarcated isolectin positive CNV. DX2 injection showed decreased vascular leakage compared to GFP injection control in fluorescein angiography (FIG. 7). Similarly, DX2 injected mice were showed reduced CNV area compared to GFP injected mice in ICG angiography (FIG. 7). Also, choroid flat mounts stained with isolectin-B4 demonstrates significant reduction in CNV formation areas in DX2 injected mice (FIG. 7).

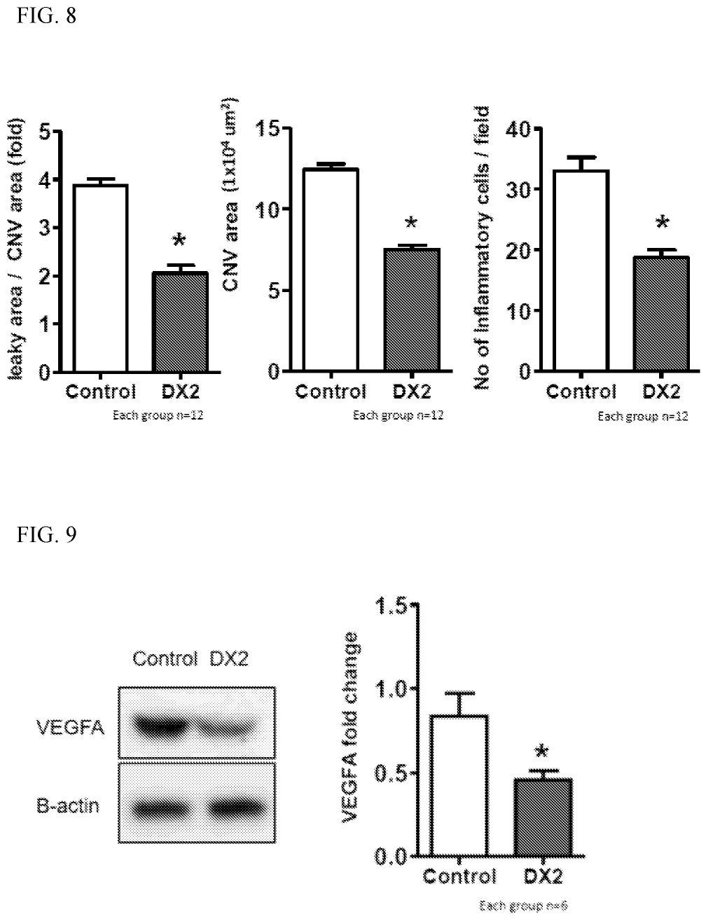

The ratio of leaky area to CNV area were estimated by measuring the total hyperfluorescent area using fluorescein angiography (FA) and the CNV area using ICGA (FIG. 8). The mean CNV area at Bruch's membrane using isolectin B4 staining were also significantly smaller in DX2 injected mice compared with that in GFP controls (n=12) (FIG. 8). Based on this result, DX2 had preventive effect in the CNV mouse model.

Inflammatory cells, in particular (macrophages), have been histologically demonstrated near/within AMD lesions, including areas of Bruch membrane breakdown, RPE atrophy, and CNV. (Macrophages) in CNV lesions have been shown to secrete proangiogenic factors such as VEGF and proinflammatory cytokines such as TNF. In FIG. 8, number of inflammatory cells of DX2 injected mice are significantly smaller compare to GFP control CNV cell.

An excessive amount of vascular endothelial growth factor (VEGF) triggers the growth and leakage of abnormal blood vessels under the macular, resulting in irreversible loss of central vision. In this context, many efforts have been made toward the development of anti-angiogenic therapies targeting VEGF for the treatment of wet AMD. These drugs have been shown to slow the progression of AMD, and in some cases, improve vision acuity by suppressing angiogenesis. Here, we treated DX2 in advance to laser-induced choroidal neovascularization mice and compared to GFP treated mice on VEGF expression (n=6) (FIG. 9). Interestingly, DX2 treated mice are showed less expression compared to GFP treated mice. This data also showed preventive effect of DX2 on CNV model mouse.

### Example 6

### Dry AMD Mouse Model

### Materials and Methods

### 6-1

### Animal Experiments

For the Dry-AMD mouse model experiment, Mdm1−/−(CRISPR/Cas9 KO) mouse present progressive photoreceptor and RPE degeneration for both dry-AMD & hereditary retinal degeneration. The animals were housed in individual cages under specific pathogen-free conditions and a constant environment condition (21° C.-23° C. temperature, 50-60% humidity and 12-h light/dark cycle) in the animal facility. AAV2-DX2 and Negative control (AAV2-GFP) injection at Sub-retinal space at 3weeks old. Histological measurements and functional recovery of retina were performed at 3-months old.

### 6-2

### Therapeutic Effect of DX2 on Dry AMD and Retinal Degeneration

Three-week-old Mdm1−/− mice were injected at subretinal space by trans-scleral injection to minimize retinal wound with AAV2-DX2/ AAV2-GFP in a volume of 4 μl using a 38G sterile micro-tip needle (INCYTO, KR). AAV2-GFP/DX2 was injected into the same animal. AAV2-GFP was injected at OS (left). AAV2-DX2 was injected at OD (right).

### 6-2-3

### Electrophysiological Function Evaluation

Three-month-old mice were used. For final measurements, WT (n=11), mdm1−/− (n=6), mdm1−/− (AAV2+GFP) (n=6) and mdm1−/− (AAV2+DX2) (n=7) were evaluated.

For electroretinogram for photoreceptor function evaluation, the OcuScience® HMsERG was used. Mice were induced to anesthesia with Avertin (1%) and anesthesia was maintained with 3% isoflurane inhalation and put on heating pad to maintain their physiological condition. A drop of 2% hypromellose solution drop was placed on the Rodent Contact Lens with Silver-embedded Thread Electrode to keep contact with the cornea and to keep it moistened. Mice were placed under the 76 mm diameter Ganzfeld dome for darkness and uniform illumination of the eyes. Measurements were performed under ISCEV-Extended full-field ERG standards protocols. The data were analyzed using ERGVIEW and the combined standard Rod&Cone response value was selected to analyze with a flash intensity of 3000 mcd·s/m², 0.10 Hz. A-wave analysis was performed for photoreceptor cell function. B-wave analysis was performed for bipolar and horizontal cell function. Amplitude and latency values for a-wave and b-wave were analyzed.

### 6-2-4

### Histological Measurements

All mice were euthanatized after ERG and their eyeballs were harvested. The eyeballs were fixed in 4% PFA overnight at 4° C. Eyeballs were dehydrated at 30% sucrose and embedded with the OCT compound for tissue cryosection. All retina cryosection samples were acquired from the optic nerve containing section with 10 μm thickness.

Retina cryosections were analyzed. H&E was used for layer thickness analysis. Layer thickness analysis was performed with Leica LAS program. Immunofluorescence was used for RPE65 and Opsin expression, and Proliferation evaluation(Ki67). Immunofluorescence ROI set and overlapping coefficient measurements were measured with Image J.

### 6-2-5

### Statistical Analysis

Student's t-test was used for primary analysis. P-values were compared with mdm1−/− (AAV2+DX2). For recovery evaluation, P values were compared to WT. For full data, Levene's Homogeneity of Variance test, ANOVA tests, and post-hoc (Dunnett (T3), Tukey HSD) evaluation were performed with IBM SPSS statistics 23.

### 6-2-6

### Results

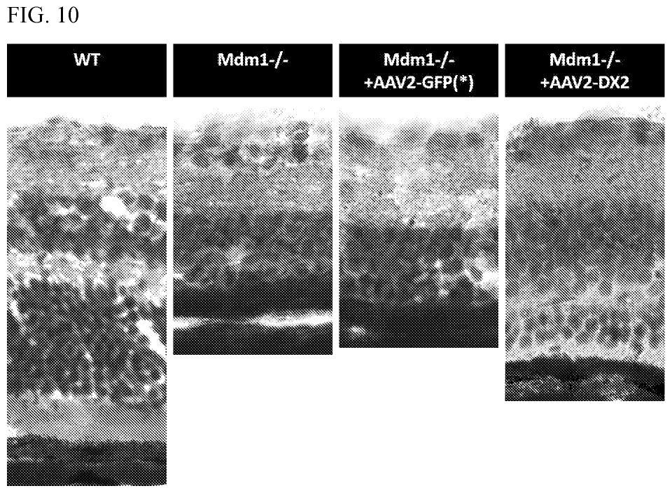

FIG. 10 shows cross-sectional histology (H&E staining) of retina.

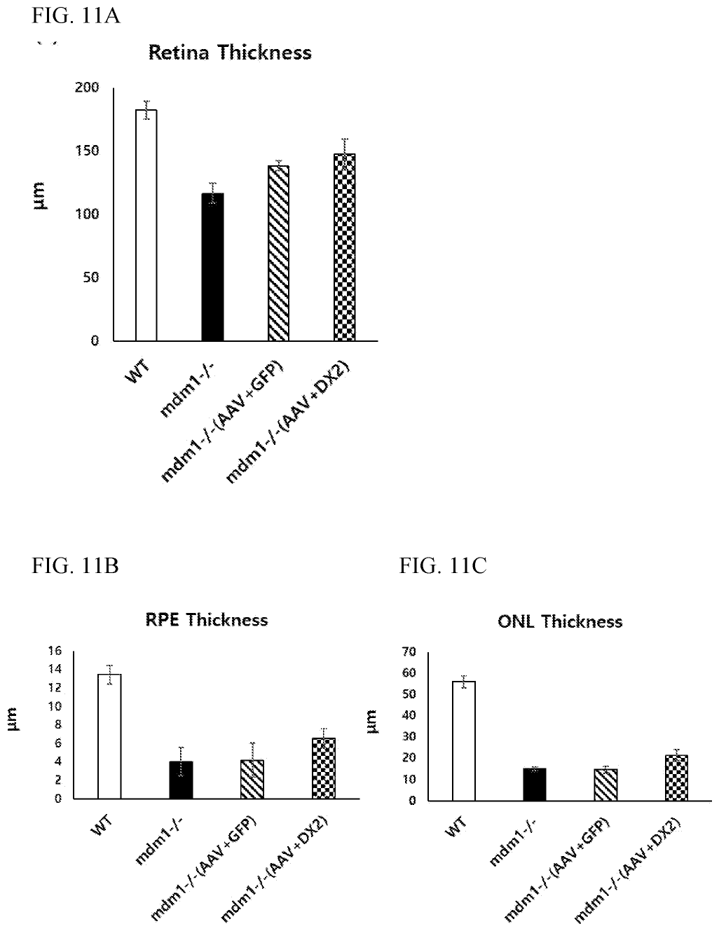

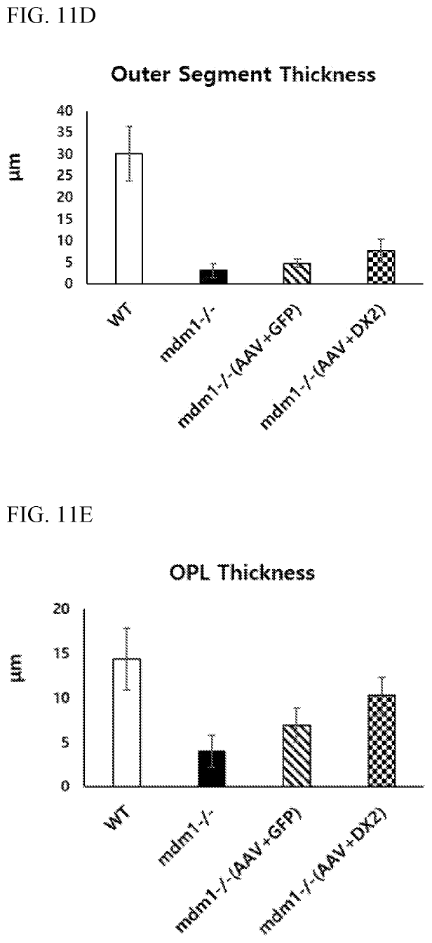

FIGS. 11A-11E show histological measurements of histological retinal thickness. FIG. 11A shows retina thickness. FIG. 11B shows RPE (Retinal Pigment Epithelial) thickness. FIG. 11C shows ONL (Outer Nuclear Layer of Photoreceptors) thickness. FIG. 11D shows Outer Segment thickness. FIG. 11E shows OPL Outer Plexiform Layer) thickness. All the samples were acquired from the optic nerve containing section with 10 μm thickness. Transfection of DX2 gene in retina resulted in the recovery of the total neural retina thickness (FIG. 11A). Transfection of DX2 showed thicker RPE layer (FIG. 11B) and the photoreceptor outer segment layer (FIG. 11C), implying that DX2 expression prevents RPE degeneration and photoreceptor cilia degradation. Transfection of DX2 also showed thicker Outer Nuclear Layer of photoreceptors (FIG. 11D) and Outer Plexiform Layer (FIG. 11E), indicating that DX2 expression reduces photoreceptor degeneration.

FIG. 12 shows integrity and proliferation of RPE (Retinal Pigment Epithelial). Transfection of DX2 gene resulted in the recovery of RPE integrity by activating proliferation of RPE.

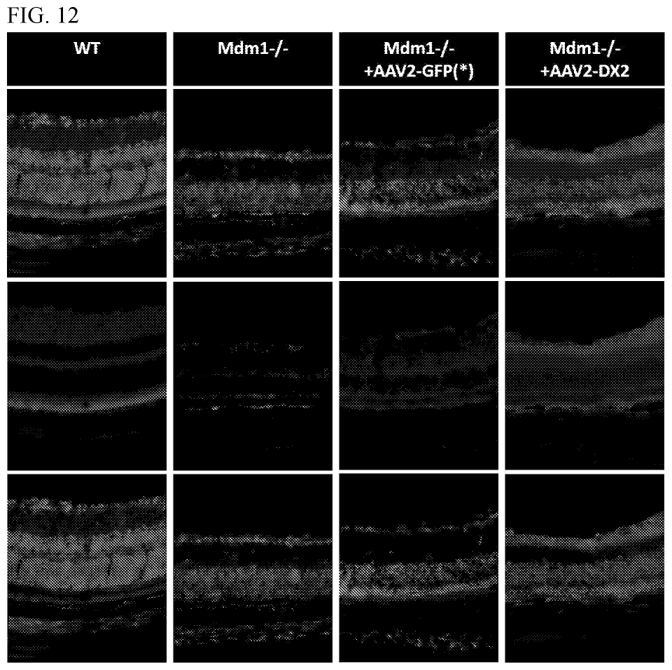

FIG. 13 shows PR (Photoreceptor) recovery. Transfection of DX2 gene resulted in recovery of PR population by activating proliferation of PR.

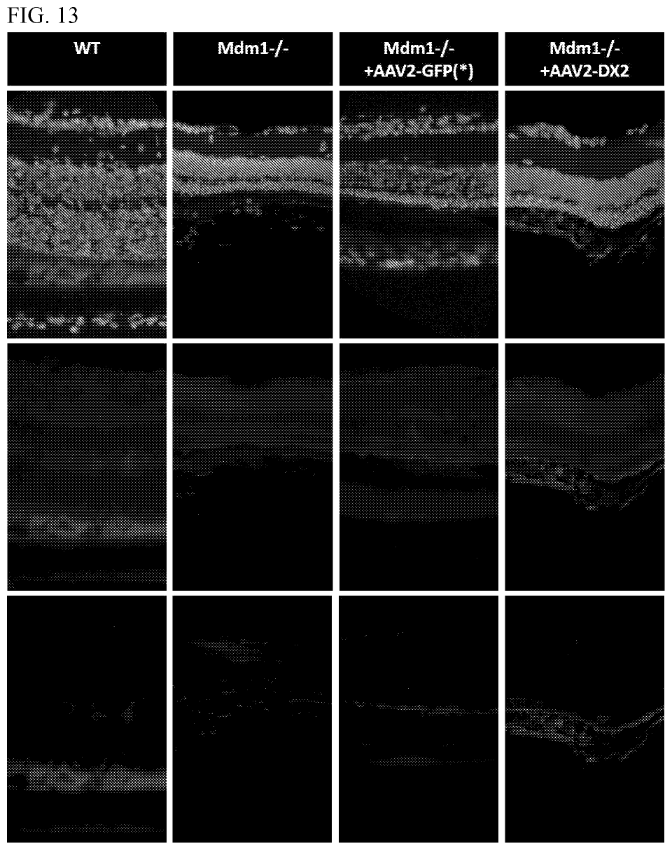

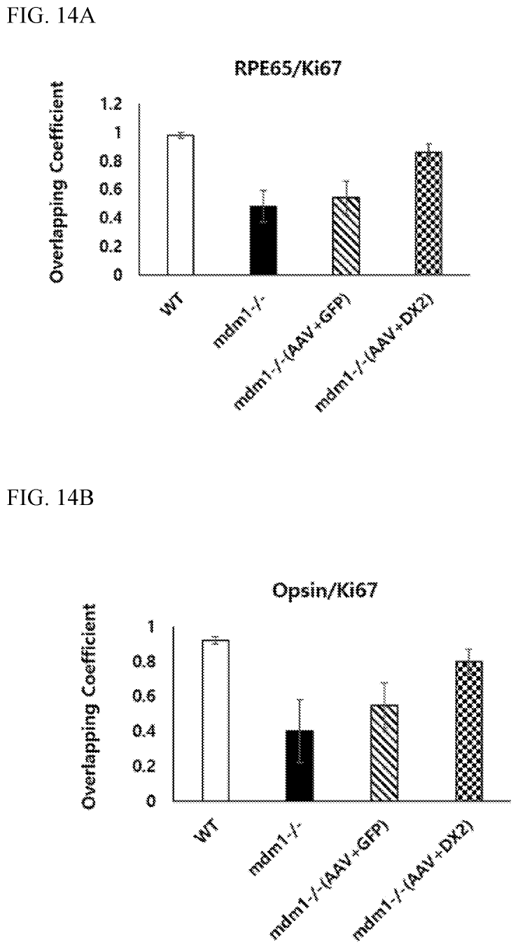

FIGS. 14A-14B show cellular proliferation of RPE and PR. Ki67 expression was measured to analyze proliferation in RPE and photoreceptor layers. Proliferation in RPE (FIG. 14A) and photoreceptor outer segment layer (FIG. 14B) was significantly higher in the AAV2-DX2 transfected sample.

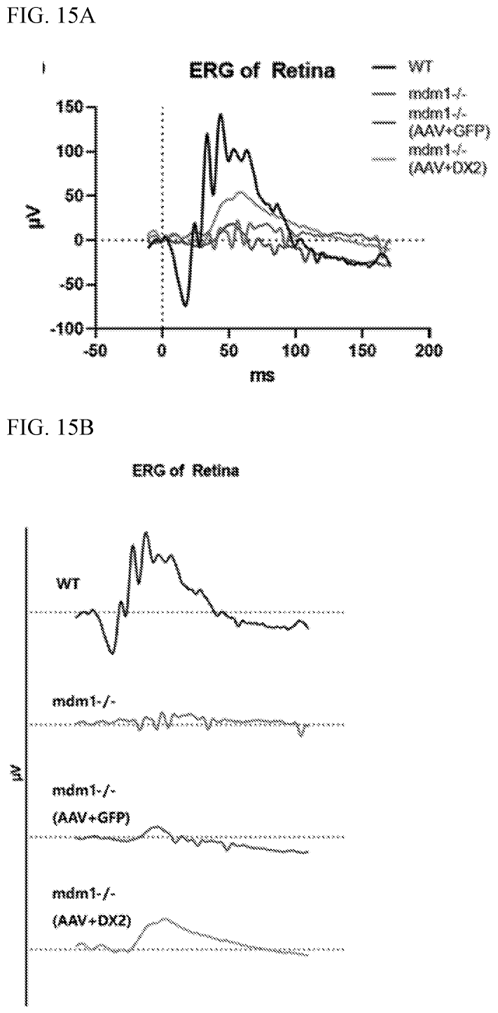

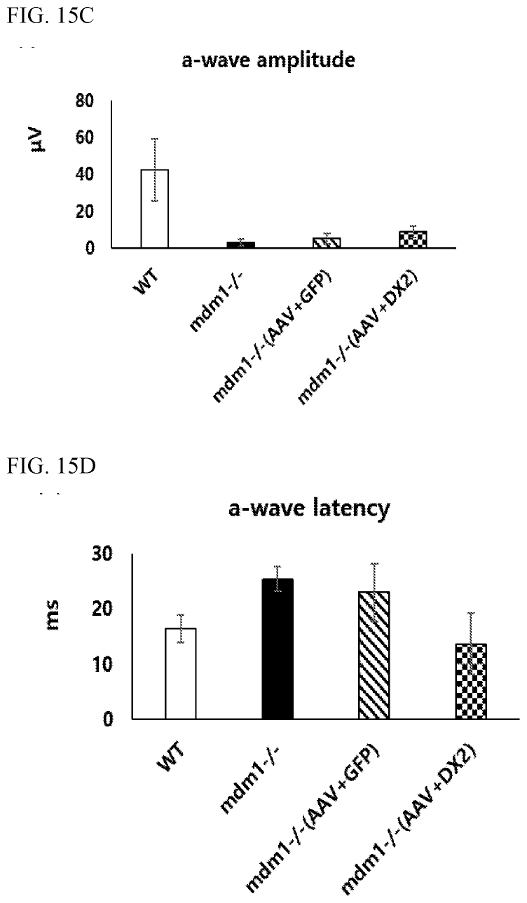

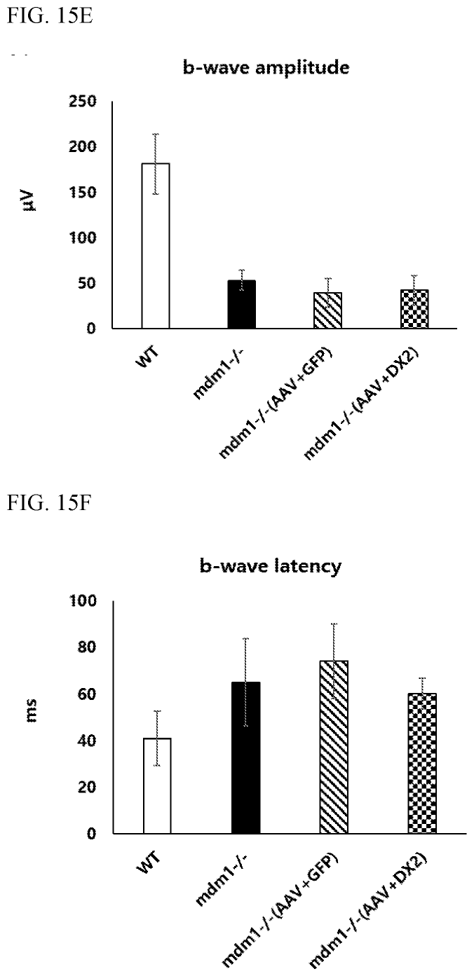

FIGS. 15A-15D show functional recovery of retina. AAV2-DX2 transfection showed increased a-wave amplitude (FIG. 15A) and reduced latency (FIG. 15B) compared to the dry-AMD model (mdm1−/−) or the negative control (mdm1−/−+AAV-GFP), indicating that DX2 expression reduces damage of photoreceptors' electrophysiological function and visual acuity. AAV2-DX2 transfected sample did not show a change in b-wave amplitude (FIG. 15C) but reduced the latency (FIG. 15D) compared to the dry AMD model (mdm1−/−), which suggests that DX2 affects only the RPE and photoreceptor, but not the bipolar cells (post-photoreceptors neurons).

Electroretinograph of AAV2-DX2 transfected sample showed increased regaining of normal ERG graph format (FIGS. 15A and 15B). AAV2-DX2 transfected sample showed slightly increased a-wave amplitude (FIG. 15C) than and reduced latency (FIG. 15D) than the dry AMD model (mdm1−/−) indicating that the DX2 expression reduced the damage of photoreceptors' electrophysiological function. AAV2-DX2 transfected sample showed did not change b-wave amplitude (FIG. 15E) but reduced the latency (FIG. 15F) than the dry AMD model (mdm1−/−).

### REFERENCES

KR 10-1067816 (2011).

Telegina, D. V., O. S. Kozhevnikova, and N. G. Kolosova. “Molecular mechanisms of cell death in retina during development of age-related macular degeneration.” Advances in Gerontology 7.1 (2017): 17-24.

Hernández-Zimbrón, Luis Fernando, et al. “Age-related macular degeneration: new paradigms for treatment and management of AMD.” Oxidative medicine and cellular longevity 2018 (2018).

Du, Hongjun, et al. “JNK inhibition reduces apoptosis and neovascularization in a murine model of age-related macular degeneration.” Proceedings of the National Academy of Sciences 110.6 (2013): 2377-2382.

Brown et al., Endogenous microRNA regulation suppresses transgene expression in hematopoietic lineages and enables stable gene transfer, Nature Med. 12:585-591 (2006).

Brown et al., Endogenous microRNA can broadly exploited to regulate transggene expression according to tissue, lineage and diffferentiation state, Nature Biotech. 25:12457-1467 (2007).

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

1. A method of treating age-related macular disease (AMD) in a subject in need thereof, comprising administering to the subject a pharmaceutically effective amount of a recombinant vector comprising an exon 2-deleted AIMP2 variant (AIMP2-DX2) gene.

2. The method of claim 1, wherein the AMD is wet AMD.

3. The method of claim 1, wherein the AMD is dry AMD.

4. The method of claim 1, wherein the vector further comprises an miR-142 target sequence.

5. The method of claim 1, wherein the vector further comprises a promoter operably linked to the AIMP2-DX2.

6. The method of claim 5, wherein the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

7. The method of claim 4, wherein the miR-142 target sequence is 3′ to the AIMP2-DX2 gene.

8. The method of claim 1, wherein the AIMP2-DX2 gene comprises a nucleotide sequence encoding an amino acid sequence that is at least 90% identical to SEQ ID NO:2, 13, 14, 15, 16, 17, 18, 19, or 20.

9. The method of claim 8, wherein the AIMP2-DX2 gene comprises a nucleotide sequence encoding an amino acid sequence of SEQ ID NO:2, 13, 14, 15, 16, 17, 18, 19, or 20.

10. The method of claim 1, wherein the AIMP2-DX2 gene does not have an exon comprising a nucleotide sequence encoding an amino acid sequence that is at least 90% identical to SEQ ID NO:10 or 11.

11. The method of claim 1, wherein the AIMP2-DX2 gene does not have an exon comprising a nucleotide sequence encoding an amino acid sequence of SEQ ID NO:10 or 11.

12. The method of claim 4, wherein the miR-142 target sequence comprises ACACTA.

13. The method of claim 4, wherein the miR-142 target sequence comprises ACACTA and 1-17 additional contiguous nucleotides of SEQ ID NO:5.

14. The method of claim 4, wherein the miR-142 target sequence comprises a nucleotide sequence at least 50% identical to a nucleotide sequence of SEQ ID NO:5 (TCCATAAAGTAGGAAACACTACA).

15. The method of claim 14, wherein the miR-142 target sequence comprises a nucleotide sequence of SEQ ID NO:5.

16. The method of claim 4, wherein the miR-142 target sequence comprises ACTTTA.

17. The method of claim 4, wherein the miR-142 target sequence comprises ACTTTA and 1-15 additional contiguous nucleotides of SEQ ID NO:7.

18. The method of claim 4, wherein the miR-142 target sequence comprises a nucleotide sequence at least 50% identical to a nucleotide sequence of SEQ ID NO:7 (AGTAGTGCTTTCTACTTTATG).

19. The method of claim 18, wherein the miR-142 target sequence comprises a nucleotide sequence of SEQ ID NO:7.

20. The method of claim 4, wherein the miR-142 target sequence is repeated 2-10 times.

21. The method of claim 1, wherein the vector is a viral vector.

22. The method of claim 21, wherein the viral vector is an adenovirus, adeno-associated virus, lentivirus, retrovirus, human immunodeficiency virus (HIV), murine leukemia virus (MLV), avian sarcoma/leukosis (ASLV), spleen necrosis virus (SNV), Rous sarcoma virus (RSV), mouse mammary tumor virus (MMTV), vaccinia virus, or Herpes simplex virus vector.

23. The method of claim 1, wherein the recombinant vector is administered topically to, by intravitreal injection to, by subconjunctival injection to, or into a subretinal space of the subject.

24. The method of claim 1, further comprising administering to the subject an additional therapeutic agent.

25. The method of claim 24, wherein the additional therapeutic agent is ranibizumab, aflibercept, or bevacizumab.


SEQUENCE LISTING (25 sequences)

SEQ ID NO: 1 - DNA, 756 bp, Artificial Sequence
  AIMP2-DX2
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctccttttaacacggccctcaagctccttaagtga

SEQ ID NO: 2 - protein, 251 aa, Artificial Sequence
  AIMP2-DX2
MPMYQVKPYHGGGAPLRVELPTCMYRLPNVHGRSYGPAPGAGHVQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 3 - DNA, 936 bp, Artificial Sequence
  AIMP2
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggaagagtctaacctgtctctgcaagctcttgagtcccgccaagatgatattttaaaacgtctgtatgagttgaaagctgcagttgatggcctctccaagatgattcaaacaccagatgcagacttggatgtaaccaacataatccaagcggatgagcccacgactttaaccaccaatgcgctggacttgaattcagtgcttgggaaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctcctttt

SEQ ID NO: 4 - DNA, 207 bp, Artificial Sequence
  Exon 2 of AIMP2
gaagagtctaacctgtctctgcaagctcttgagtcccgccaagatgatattttaaaacgtctgtatgagttgaaagctgcagttgatggcctctccaagatgattcaaacaccagatgcagacttggatgtaaccaacataatccaagcggatgagcccacgactttaaccaccaatgcgctggacttgaattcagtgcttgggaag

SEQ ID NO: 5 - DNA, 23 bp, Artificial Sequence
  miR-142-3p target sequence
tccataaagtaggaaacactaca

SEQ ID NO: 6 - DNA, 132 bp, Artificial Sequence
  4 repeats of miR-142-3p target sequence
ccagaagcttgctagctccataaagtaggaaacactacatcactccataaagtaggaaacactacagatatctccataaagtaggaaacactacatcactccataaagtaggaaacactacaaagcttgtag

SEQ ID NO: 7 - DNA, 21 bp, Artificial Sequence
  miR-142-5p target sequence
agtagtgctttctactttatg

SEQ ID NO: 8 - DNA, 24 bp, Artificial Sequence
  AIMP2 variant forward primer
ctggccacgtgcaggattacgggg

SEQ ID NO: 9 - DNA, 20 bp, Artificial Sequence
  AIMP2 variant reverse primer
aagtgaatcccagctgatag

SEQ ID NO: 10 - protein, 69 aa, Artificial Sequence
  Exon 2 of AIMP2
EESNLSLQALESRQDDILKRLYELKAAVDGLSKMIQTPDADLDVTNIIQADEPTTLTTNALDLNSVLGK

SEQ ID NO: 11 - protein, 36 aa, Artificial Sequence
  Exon 2 of AIMP2 variant
MIQTPDADLDVTNIIQADEPTTLTTNALDLNSVLGK

SEQ ID NO: 12 - protein, 320 aa, Artificial Sequence [sequence omitted]

SEQ ID NO: 13 - protein, 243 aa, Artificial Sequence
  AIMP-DX2 variant 1
MPMYQVKPYHGGGAPLRVELPTCMYRLPNVHGRSYGPAPGAGHVQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPF

SEQ ID NO: 14 - protein, 244 aa, Artificial Sequence
  AIMP2-DX2 variant 2
MQMEGTAHVKICGQSQGGGLGTPRTVWLEHRQRTKLGEDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 15 - protein, 207 aa, Artificial Sequence
  AIMP2-DX2 variant 3
MDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 16 - protein, 222 aa, Artificial Sequence
  AIMP2-DX2 variant 4
MNSPAVNTLIQRSRHGDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 17 - protein, 211 aa, Artificial Sequence
  AIMP2-DX2 variant 5
MPMYQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 18 - protein, 251 aa, Artificial Sequence; identical to SEQ ID NO: 2
  AIMP2-DX2 variant 6

SEQ ID NO: 19 - protein, 206 aa, Artificial Sequence
  AIMP2-DX2 variant 7
DYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 20 - protein, 198 aa, Artificial Sequence
  AIMP2-DX2 consensus sequence
DYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPF

SEQ ID NO: 21 - DNA, 16 bp, Artificial Sequence
  Nhe1, Hind III, and Bmt1 sites in miR-142-3pT with 4 repeats
ccagaagcttgctagc

SEQ ID NO: 22 - DNA, 10 bp, Artificial Sequence
  Hind H site in miR-142-3pT with 4 repeats
aagcttgtag

SEQ ID NO: 23 - RNA, 23 nt, Artificial Sequence
  miR-142-3p
uguaguguuuccuacuuuaugga

SEQ ID NO: 24 - RNA, 21 nt, Artificial Sequence
  miR-142-5p
cauaaaguagaaagcacuacu

SEQ ID NO: 25 - DNA, 238 bp, Artificial Sequence
  miR-142-3pT target sequence mutant
ccgctgcagtgtgacagtgccagccaatgtgcagaggtggatgaggtcttgtgaaaacctggctccttttaacacggccctcaagctccttaagtgaccagaagcttgctagctccataaagtaggaccactgcaatcactccataaagtaggaccactgcaagatatctccataaagtaggaccactgcaatcactccataaagtaggaccactgcaaaagcttgtagggatccgcc